(19) United States
(12) Patent Application Publication
(10) Pub. No.: US 2021/0263971 A1
(43) Pub. Date: Aug. 26, 2021

(54) AUTOMATIC CORPORA ANNOTATION

(71) Applicant: Health Care Service Corporation, a Mutual Legal Reserve Company, Chicago, IL (US)

(72) Inventor: Paul Landes, Oak Park, IL (US)

(73) Assignee: Health Care Service Corporation, a Mutual Legal Reserve Company, Chicago, IL (US)

(21) Appl. No.: 16/800,782

(22) Filed: Feb. 25, 2020

(51) Int. Cl. G06F 16/908; G16H 10/60; G06F 16/903; G06F 16/33; G06N 7/00; G06N 20/00

(52) U.S. Cl. CPC G06F 16/908; G16H 10/60; G06N 20/00; G06F 16/3346; G06N 7/005; G06F 16/90344

(57) ABSTRACT

A computer implemented method and system for automatically creating an annotated dataset. An automatic annotating system may access a proprietary database and an unannotated dataset and identify tokens, or character spans, of the unannotated dataset that match property values in the database. The automatic annotating system may then determine whether the identified tokens in the unannotated dataset originated, or derived, from the database by calculating probabilities using a language model and a Bayesian network. The automatic annotating system annotates identified tokens determined to originate from the database by associating a tag to each identified token and assigning annotation attributes for each tag. The annotations and associated properties and values are stored as an annotated dataset. The annotated dataset may then be used train automated, machine learned models to identify and tag other datasets.

### BACKGROUND

Medical data, or electronic health records (EHR's), can be used to develop and advance medical science. Documents, such as EHR's, having textual descriptions of patient medical records contain an abundance of useful information, such as disease treatment and medical information. This type of information has been recognized as an important component of clinical studies and decision-making medical applications. This recognition has led to an increased use of medical data in medical research, which has led to an increased risk of exposure of protected health information (PHI). PHI, as defined by the Health Insurance Portability and Accountability Act (HIPAA) of 1996, is any information about health status, provision of health care, or payment for health care that is created or collected by a Covered Entity (or a Business Associate of a Covered Entity) and can be linked to a specific individual. This definition is typically interpreted rather broadly and may include any part of a patient's medical record or payment history. Health information such as diagnoses, treatment information, medical test results, and prescription information are considered PHI, as are national identification numbers and demographic information such as birth dates, gender, ethnicity, and contact and emergency contact information. PHI may not only include medical data, but may also include personally identifiable information (PII) as well. Examples include disease carriers, medical record numbers, social security numbers and all other personal identification information.

With the increased use of EHR's, protecting private information that may potentially be disclosed has become a major concern for healthcare providers and medical researchers. Protecting patient identity and confidentiality is vital when using medical data for analysis, and exposure of PHI adds tremendous risk to patients, providers and the health care industry. To protect patient confidentiality and privacy and facilitate the use and dissemination of patient specific EHR's, and to avoid the need for obtaining individual patient consent before using medical records, PHI needs to be extracted from clinical data before use. This can be done by de-identification or anonymization.

De-identification is the process of identifying and removing or replacing the confidential or sensitive information in the data while keeping the rest of the data otherwise intact. Under the safe harbor provision of HIPAA, de-identification occurs when specified identifiers of the patient, and of the patient's relatives, household members, and employers, are removed such that, after the removal of the specific identifiers, the Covered Entity (or a Business Associate of a Covered Entity) has no actual knowledge, e.g. based on the remaining information, that could be used to identify the patient. De-identified data may be coded with a link to the original, fully identified data set, which makes de-identified data considered indirectly identifiable. Anonymization, on the other hand, is a process in which PHI elements are eliminated or manipulated with the purpose of hindering the possibility of going back to the original data set.

PHI is often sought out in both structured and unstructured datasets for de-identification or anonymization before disclosing the dataset in order to preserve privacy, such as may be required by legal, regulatory, industry and/or ethical regulations, requirements or guidelines. For example, researchers may wish to remove PHI from a dataset before sharing the dataset publicly. In another example, an organization may want to mask PHI before sending a dataset containing PHI to a third party for developing or testing purposes. Finding instances of PHI in text is mainly an exercise in data mining, where the goal is to identify instances of specific PHI data types, such as patient names, ages, genders, addresses, or social security numbers. This process can be extremely challenging, particularly for human annotators who manually search for the instances of PHI in large datasets.

Efforts to automatically identify and remove PHI has been a challenge and the subject of much work since HIPAA. Recent applications of machine or deep learning methods to linguistic techniques have become popular both in academia and in industry. Automated, machine learning or artificial intelligence-based systems or models may exist for identifying PHI in electronic documents, such as, for example, to recognize particular character spans in the text as PHI. However, such automated systems must first be trained using annotated corpora (text based data sets). An annotated corpus may be a body of sample text that has been pre-annotated in a machine-identifiable manner, e.g. with “tags,” identifying examples of PHI. These systems or models need a large number of training examples to perform well and achieve accurate results. However, one problem is a lack of enough data containing PHI to form the necessary corpora to be trained on. Another problem is that manually annotating large corpora sufficient to train these systems is not feasible.

### BRIEF DESCRIPTION OF THE DRAWINGS

FIGS. 1A-B depict an exemplary system for automatically annotating a dataset of an authorized computer system according to one embodiment.

FIG. 2 depicts an exemplary flow chart for the disclosed Bayesian inference process according to one embodiment.

FIG. 3 depicts a flow chart illustrating an exemplary operation of the system of FIGS. 1A-1B.

FIG. 4 shows an illustrative embodiment of a specialized computer system configured for automatically annotating a dataset of an authorized computer system.

FIG. 5 illustrates an exemplary relationship between database items, properties, and values.

FIG. 6 illustrates an exemplary annotated document segment from a corpus.

FIG. 7 illustrates annotations to the exemplary document segment of FIG. 6.

FIG. 8 depicts a flow chart illustrating an overview of an exemplary process.

FIG. 9 depicts a flow chart illustrating an overview of creating an exemplary Bayesian network.

FIG. 10 illustrates an exemplary Bayesian network.

FIG. 11 illustrates an exemplary use case of the disclosed framework for selecting an item node of the Bayesian network of FIG. 10.

FIG. 12 illustrates an exemplary use case of the disclosed framework for observing an item node of the Bayesian network of FIG. 10.

FIG. 13 illustrates an exemplary use case of the disclosed framework for selecting and observing property nodes of the Bayesian network of FIG. 10.

### DETAILED DESCRIPTION

The disclosed embodiments relate to a system and method to automatically create a large annotated corpus, or text-based data set, of both structured and unstructured data which may then be used to train automated, machine learning, or artificial intelligence-based systems/models for identifying PHI in other data sets. The disclosed embodiments automatically create an annotated corpus by detecting spans of text in an unannotated corpus that match literal values in a database (i.e., a store of data or knowledge base) and then annotating those spans of text, the annotations being information stored in a data structure with an association to one or more data items.

As used herein, annotating may refer to the act of assigning tags to text strings, as will be discussed below, and the framework disclosed herein that annotates, or tags, an unannotated corpus may be referred to as a “tagger,” which demarcates and assigns text spans with probabilities of the likelihood that a given text span belongs to a particular entity. The disclosed embodiments may be based on a fundamental assumption that anything that can and should be annotated originates from a database, such as a database which contains medical related information, including PHI, and the unannotated corpus is either generated or contains verbatim mentions from that database.

More particularly, the disclosed embodiments may first pre-process the database to identify and assign data types contained therein and determine probabilities of the data therein appearing in the corpus. The disclosed embodiments may then pre-process the corpus to parse spans of characters, e.g. segment the data into tokens, such as words, phrases, numbers, etc., to determine candidate tokens, or character spans, to be checked in the database. Candidate tokens are tokens considered for annotation, and each token from the corpus that is found in the database is considered for annotation. The number of candidate tokens to check against the database may be reduced to a manageable number using known techniques. If a candidate token is not found in the database, the process ends for that token. For candidate tokens which are found in the database, the disclosed embodiments recognize that some data appearing in the corpus might match data contained in the database despite not having been derived from the database, e.g. it may have come from another source. As the disclosed embodiments may not know for sure how such data arrived to the corpus, it must analyze and compute the probabilities that the data came from the database to know if the database property for that data applies.

To determine whether data from the corpus derived from the database, the disclosed embodiments may use a known language model along with a Bayesian network (a probabilistic graph structure) to calculate probabilities of whether candidate tokens originated from the database. A Bayesian network is a graphical model used to robustly represent a configuration of random variables as conditional probabilities. Specifically, Bayesian networks are a type of probabilistic graphical model that uses Bayesian inference for probability computations. A Bayesian network models a conditional probability distribution of a set of random variables with a possible mutual causal relationship. The network consists of nodes of a graph representing the random variables, edges between pairs of nodes representing the causal relationship of these nodes, and a conditional probability distribution in each of the nodes. The conditional probability may be stored and represented in a conditional probability table (CPT). The main objective of the method of a Bayesian network is to model the posterior conditional probability distribution of outcome (often causal) variable(s) after observing certain evidence, such as observing a node of the Bayesian network. As used herein, observing a node refers to maximizing the probability of a state on a particular Bayesian network node (i.e., setting the probability of a state on a particular Bayesian network node to one (1)). Observing a node is a process by where one state of a random variable, and thus one row in the CPT, is given a probability of one (1) (100%) and all other states 0 (0%).

The disclosed embodiments may seek to annotate identified character spans which likely originated from the database with the property value of that data from the database. For each annotation of a candidate token found in the database, the disclosed embodiments may assign a tag that reflects at least the probability that the annotated token was derived from the database and the database property thereof. Once the tags are created, a Bayesian network is constructed. A CPT is computed for each node of the Bayesian network during the construction of the graph of the Bayesian network. The Bayesian network may then be used to infer tag probabilities for each combination of item and property.

Conventional methods for identifying and tagging PHI may include automated and machine or deep learning methods, as well as linguistic techniques. However, performance using these conventional techniques is dependent on large corpora needed to train classifiers. The necessary training, however, is performed manually, which is cumbersome, time consuming, and prone to errors. Further, the amount of necessary annotated data needed in order to train conventional systems may not be available.

The disclosed embodiments differ from these conventional systems. In particular, in the disclosed embodiments, the base unannotated corpus of structured and unstructured text is known to contain PHI. For example, for the disclosed embodiments, the unannotated corpus is created from a large collection of medical related information, e.g. emails, medical records, reports, doctor's notes, etc. This can be done only by entities which are already privy to or otherwise authorized to possess such information under current regulatory guidelines. Examples of such entities include healthcare providers, healthcare facilities, and health insurers. The disclosed embodiments also use a database known to contain PHI data in a structured form. Again, this “proprietary” database can only be provided by entities which are already privy to or otherwise authorized to possess such information under current regulatory guidelines, such as the exemplary entities mentioned above. An example of such a database is a medical insurance claims database maintained by a health insurer. This proprietary database provides a basis to the disclosed embodiments defining what is and is not PHI. For example, the database may be structured into fields, such as name, age, address, etc. Where data in the unannotated corpus is determined to match data in the database, e.g. when a span of characters matches a name in the database, the disclosed embodiments may then tag that character span as a “name.” The proposed process assumes that at least some PHI contained in the unannotated corpus was derived from the database. Deriving, or originating, from the database may mean that the data first existed in the database and then was copied to the unannotated corpus, e.g. copied to a note or a file contained therein. For example, an email reporting a medical test result to a patient may be assumed to have been generated by a system which accessed data from the database. It is noted that even if an entity other than those described above has access to some PHI, e.g. via fabrication or otherwise getting patients to opt-in, etc., such entities may not have access to a sufficient volume of such data to implement the disclosed embodiments.

The disclosed embodiments provide a specific manner of automatically annotating data by providing data and determining whether identified data in one source represents data from another source based on probabilities calculated using a natural language model and Bayesian network, which provides a specific technical improvement over prior systems resulting in an improvement in computer functionality, e.g., the use of a natural language model along with a Bayesian network to automatically determine whether identified data in a corpus originated from a proprietary database is a specific improvement in computer functionality over prior art systems by automating and rendering computerized functions of identifying and tagging certain types of data more efficient. The processing rules of the disclosed embodiments tied to the automation of annotating a large corpus solves a technical problem of annotating large amounts of data in an efficient and reliable manner, which is not feasible to do manually.

The exemplary framework disclosed herein is unique in that it solves the problem of having an inadequate volume of data (i.e., annotated corpora) needed to train conventional PHI identification systems. Untapped sources of data include health care industry data and natural language text that includes PHI. The proposed system also improves the manner in which the necessary amount of training data is annotated, since manually training conventional systems to annotate enough corpora is not feasible. The proposed system automatically annotates large amounts of existing corpora that are then used to train automated, machine learned systems or models to identify and tag PHI, which increases efficiency, decreases costs and time associated with software training, and improves overall internal business processes. This provides a specific technical improvement over prior systems, resulting in an improved data annotation system.

The ability for entities which are already privy to or otherwise authorized to possess data such as PHI under current regulatory guidelines, such as healthcare providers, healthcare facilities, and health insurers, to use their existing, proprietary sources of data that are known to contain PHI to automatically create an annotated corpus used to train other systems may improve the ability for these entities to more accurately and efficiently identify PHI in order to de-identify or anonymize the PHI, which is an ongoing business concern for these entities that need to comply with privacy regulations such as HIPPA or the General Data Protection Regulation (GDPR). Furthermore, the usage of the disclosed methods and systems may enable software development teams and teams concerned with dealing with PHI to not have to rely on manually identifying and annotating data containing PHI in order to create training corpora, which may be a common approach to creating training data sets in PHI identification systems. Teams can use existing data known to contain PHI to reduce the risk of exposure of such PHI, sensitive personal identifying information (PII), or other sensitive and regulated data.

The present disclosure provides an improved method and system for automatically annotating data, which may reduce cost and time associated with software training, increase efficiency, and improve accuracy of correctly identifying PHI. The disclosed embodiments thus provide significantly more than abstract ideas (e.g., mathematical concepts, certain methods of organizing human activity, and mental processes), laws of nature, or natural or physical phenomena, since the proposed embodiments involve methods and techniques that are more than what is well-understood, routine, or conventional activity in the field of annotating data. Further, any abstract ideas, laws of nature, or natural/physical phenomena present in this disclosure, if at all, are simply applied, relied on, or used by the proposed embodiments as an integration into a practical application of creating a training data set by automatically annotating data, such as a data set used to train machine learning or artificial intelligence-based systems or models for identifying PHI in electronic documents.

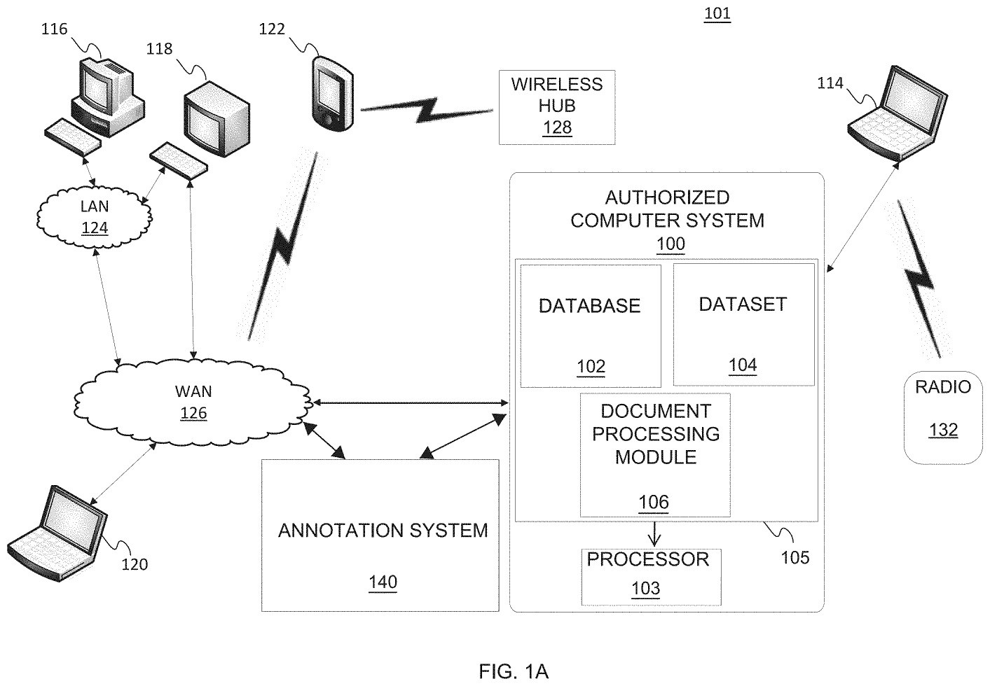

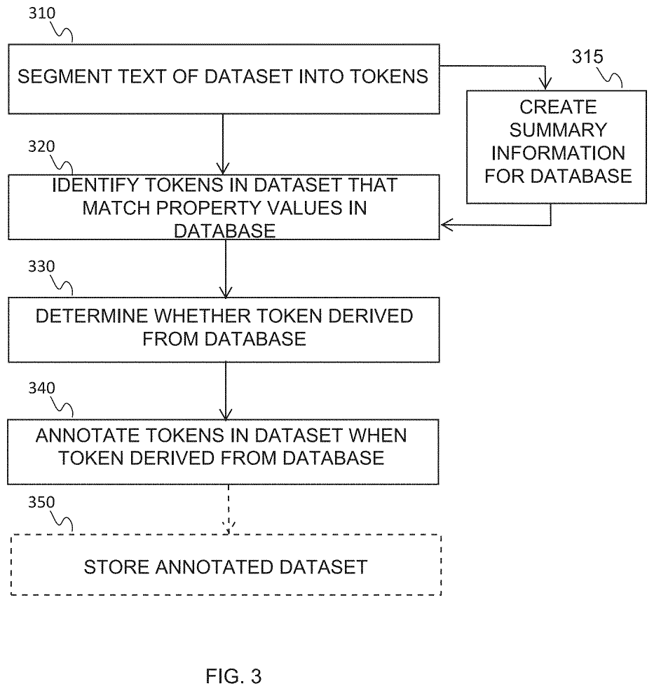

In accordance with aspects of the disclosure, systems and methods are disclosed for automatically annotating a dataset, and in particular, automatically annotating a dataset when text in the dataset that matches property values in a database is determined to be derived from the database. The disclosed embodiments generally determine whether text of the dataset is derived from the database by calculating probabilities that the text originated from the database using a language model and a Bayesian network, as described herein. The disclosed embodiments are preferably implemented with computer devices and computer networks, such as those described with respect to FIGS. 1A, 1B, and 4, that allow users, e.g. business employees, customers and parties related thereto, to automatically create annotated training corpora used to identify PHI in electronic documents.

An exemplary network environment 101 for automatically annotating a dataset 104 of an exemplary authorized computer system 100 is shown in FIG. 1A. An authorized computer system 100, such as, for example, a computer system 100 of a healthcare provider, healthcare facility, or health insurer, may receive, transmit, and/or store electronic documents of the dataset 104 between users, such as via wide area network 126 and/or local area network 124 and computer devices 114, 116, 118, 120 and 122, as will be described below, coupled with the authorized computer system 100. The electronic documents of the dataset 104 received, transmitted, and/or stored by the authorized computer system 100 may be a plurality of electronic documents and may include, for example, emails, reports, records, and/or notes in electronic form, and may contain information relating to a patient or a plurality of patients, including PHI. The electronic documents of the dataset 104 contain at least some data derived from (i.e., originated from) a database 102 of the authorized computer system 100, or contain verbatim mentions of data from the database 102. For example, a document processing module 106 of the authorized computer system 100 may access data from the database 102, generate a document therefrom, and store the document in the dataset 104. The exemplary network environment 101 shown in FIG. 1A also includes an annotation system 140 that operates to annotate the dataset 104 of the network-connected authorized computer system 100. In particular, the annotation system 140 may annotate documents contained in the dataset 104 when the annotation system 140 determines that data in the documents of the dataset 104 are representative of data contained in the database 102 of the authorized computer system 100. Further, the authorized computer system 100 may be operable to facilitate messaging or other communication between the annotation system 140 and/or the computer devices 114, 116, 118, 120 and 122 via wide area network 126 and/or local area network 124, particularly as it relates to information relating to the annotating by the annotation system 140.

In the exemplary embodiment shown in FIG. 1A, the annotation system 140 is separate and distinct from the authorized computer system 100. In another embodiment, the annotation system 140 may be incorporated as an individual module within the authorized computer system 100.

Herein, the phrase “coupled with” is defined to mean directly connected to or indirectly connected through one or more intermediate components. Such intermediate components may include both hardware and software based components. Further, to clarify the use in the pending claims and to hereby provide notice to the public, the phrases “at least one of <A>, <B>, . . . and <N>” or “at least one of <A>, <B>, . . . <N>, or combinations thereof' are defined by the Applicant in the broadest sense, superseding any other implied definitions herebefore or hereinafter unless expressly asserted by the Applicant to the contrary, to mean one or more elements selected from the group comprising A, B, . . . and N, that is to say, any combination of one or more of the elements A, B, . . . or N including any one element alone or in combination with one or more of the other elements which may also include, in combination, additional elements not listed.

The authorized computer system 100 may be implemented as a separate component or as one or more logic components, such as on an FPGA that may include a memory 105 or reconfigurable component to store logic and a processing component to execute the stored logic, or as computer program logic, stored in the memory 105, or other non-transitory computer readable medium, and executable by a processor 103, such as the processor 402 and memory 404 described below with respect to FIG. 4. In one embodiment, the system 100 is implemented by a server computer, e.g. a web server, coupled with one or more client devices 114, 116, 118, 120, 122, such as computers, mobile devices, etc. via a wired and/or wireless electronic communications network, such as the wide area network 126, local area network 124, and/or radio 132, in a network environment 101. In one embodiment, client devices 114, 116, 118, 120, 122 interact with the system 100 of the server computer to provide inputs thereto and receive outputs therefrom as described herein. The authorized computer system 100 may also be implemented with one or more mainframe, desktop or other computers, such as the computer 400 described below with respect to FIG. 4.

A database 102 or data structure may be provided which includes data identifying or relating to a patient, such as names, ages, genders, addresses, social security numbers, medical record numbers, account numbers or identifiers, usernames, passwords, and all other personal identification information. The database 102 may also include diagnoses, treatment information, medical test results, prescription information, a preferred contact method, contact information for the preferred contact method, types of insurance, codes indicating health conditions, codes indicating procedures provided by a health care provider, types of benefits covered, costs of procedures provided by a health care provider, dates of service (i.e., when the procedures were performed by the health care provider), payment details, etc. Since at least some of the information contained in the database 102 is considered PHI, possession and use of the database 102 may only be authorized to certain entities under regulatory guidelines and/or privacy regulations such as HIPPA or the GDPR. For example, some such authorized entities may include healthcare providers, healthcare facilities, medical laboratories, health insurers, medical researchers, and their affiliates for whom this data is shared.

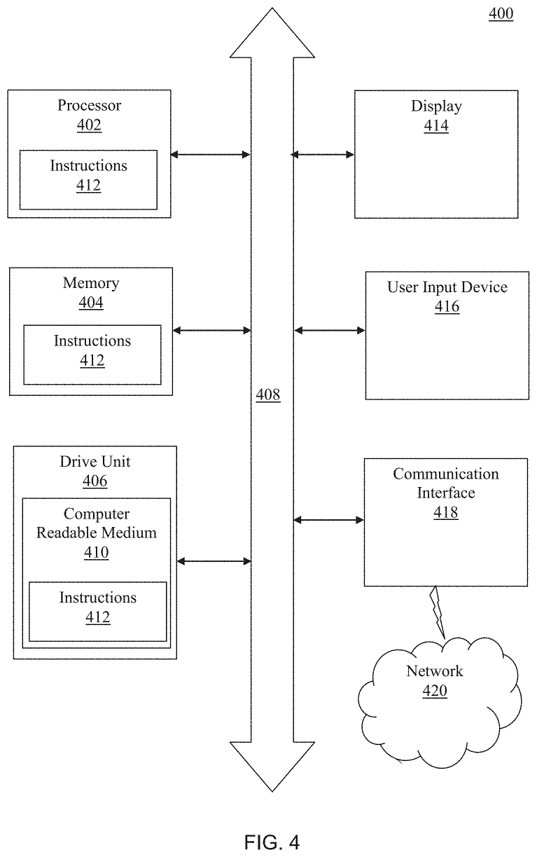

It will be appreciated that the database 102 may be stored in a memory 105 or other non-transitory medium coupled with the authorized computer system 100 and may be implemented by a plurality of databases, each of which stores a portion of the information. The database 102 is structured with a pre-defined data model or format, such as a relational database having a relational model of data. The database 102 includes a plurality of data items, or entities, stored in association with one or more properties. Each property is associated with a value. The relation from an item to a property is a one to many, with each of the latter having an associated value in the database 102. In a Relational Database Management System (RDBMS), which is a software system used to maintain relational databases, an item is a row and a property is a related value of the item in a column of the database 102. Stated another way, a property is an entity's data value, or the data instances themselves. A name space may be incorporated into the name of a property since there is no hierarchy to them. For example, the property “patient.age” could represent a column “age” in a “patient” table found in an RDBMS. The database 102 items, properties, and values will be discussed in further detail below with respect to FIG. 5.

A document processing module 106 may be provided and may be implemented as a separate component or as one or more logic components, e.g. first logic, such as on an FPGA that may include a memory 105 or reconfigurable component to store logic and a processing component to execute the stored logic, or as computer program logic, stored in the memory 105, or other non-transitory computer readable medium, and executable by a processor 103, such as the processor 402 and memory 404 described below with respect to FIG. 4, to cause the processor 103 to, or otherwise be operative to, access data from the database 102 and generate a document to be stored in the dataset 104, such as an email, medical report, etc. For example, an email reporting a medical test result to a patient may be generated by the document processing module 106, which accessed data from the database 102, and stored as part of the dataset 104. In this example, the information contained in the email (e.g., patient name, medical test results, etc.) is data that originated from the database 102. In other words, the data in the email of the dataset 104 was derived from the database 102. In another example, the document processing module 106 may be used to generate a document that mentions a person's name and number of children. In this example, the document is also stored as part of the dataset 104, but the document processing module 106 did not access the database 102 in order to generate the document. As will be discussed further below, even though the specific values of certain data items, such as the person's name and number of children in the current example, may match data values in the database 102, the values of these data items may not have originated in the database 102. In other words, the exemplary document that mentions a person's name and number of children may not have been generated based on, or from, the database 102.

It will be appreciated that documents generated, or derived, from the database 102 may be generated by sources other than the document processing module 106. For example, the document processing module 106 of the authorized computer system 100 may receive a document containing data derived from the database 102 from another authorized computer system, such as computer devices 114, 116, 118, 120 and 122, via wide area network 126 and/or local area network 124, and transfer the received document into the dataset 104. In another embodiment, the document received from another authorized computer system, such as computer devices 114, 116, 118, 120 and 122, via wide area network 126 and/or local area network 124, may be transferred directly into the dataset 104 without being processed by the document processing module 106.

A dataset 104 may be provided and may contain a large collection of electronic documents such as emails, reports, records, notes, etc. The electronic documents of the dataset 104 include text and contain at least some data derived from (i.e., originated from) the database 102 of the authorized computer system 100 or contain verbatim mentions of data from the database 102. In one embodiment, the documents of the dataset 104 may be structured with a pre-defined data model or format. In another embodiment, the documents of the dataset 104 may be unstructured. In yet another embodiment, the dataset 104 may contain both structured and unstructured documents. Structured data is data in a defined format, or code, that makes it easily readable and/or searchable by a computer. Examples of structured data include JavaScript Object Notation (JSON), Extensible Markup Language (XML) formatted files, YAML Ain't Markup Language (YAML) and fixed width/field file formats. Unstructured data is not structured with pre-defined data models or schema. Examples of unstructured data may include the content of documents, journals, books, health records, metadata, audio, video, analog data, images, files, and unstructured text such as the body of an e-mail message, Web page, or word-processor document. Besides the difference between how structured or unstructured data is stored in a relational database versus stored outside of one, the biggest difference is the ease of analyzing structured data versus unstructured data. Mature analytics tools exist for structured data, but analytics tools for mining unstructured data may be emerging and developing. Dealing with unstructured data is important, since a vast majority (i.e., 80% or higher) of all potentially usable business information may originate in unstructured form.

As discussed above, the electronic documents of the dataset 104 may be generated by the document processing module 106 or may be received from another authorized computer system, such as computer devices 114, 116, 118, 120 and 122, via wide area network 126 and/or local area network 124. The electronic documents of the dataset 104 may include a plurality of electronic documents and may contain information relating to a patient or a plurality of patients. Since, as discussed above, at least some of the data contained in the database 102 is considered PHI, and since at least some data of the dataset 104 is derived from the database 102 or contain verbatim mentions of data from the database 102, a portion of the data in the dataset 104 may also contain PHI. Thus, possession and use of the dataset 104 may also only be authorized to certain entities under regulatory guidelines and/or privacy regulations such as HIPPA or the GDPR. As discussed above, some such authorized entities may include healthcare providers, healthcare facilities, health insurers, and medical researchers. Thus, both the database 102 and the dataset 104 may be proprietary to an authorized entity, such as the authorized computer system 100.

The dataset annotating network environment 101 shown in FIG. 1A includes exemplary computer devices 114, 116, 118, 120, 122, which depict different exemplary methods or media by which a computer device may be coupled with the authorized computer system 100 or by which a user may process or communicate, e.g. send and receive, electronic documents or other information therewith. It will be appreciated that the types of computer devices deployed by users and the methods and media by which they communicate with the authorized computer system 100 is implementation dependent and may vary and that not all of the depicted computer devices and/or means/media of communication may be used and that other computer devices and/or means/media of communications, now available or later developed may be used. Each computer device, which may comprise a computer 400 described in more detail below with respect to FIG. 4, may include a central processor that controls the overall operation of the computer and a system bus that connects the central processor to one or more conventional components, such as a network card or modem. Each computer device may also include a variety of interface units and drives for reading and writing data or files and communicating with other computer devices and with the authorized computer system 100. Depending on the type of computer device, a user can interact with the computer with a keyboard, pointing device, microphone, pen device or other input device now available or later developed.

An exemplary computer device 114 is shown directly connected to the authorized computer system 100 in FIG. 1A, such as via a T1 line, a common local area network (LAN) or other wired and/or wireless medium for connecting computer devices, such as the network 420 shown in FIG. 4 and described below with respect thereto. The exemplary computer device 114 is further shown connected to a radio 132. The user of radio 132, which may include a cellular telephone, smart phone, or other wireless proprietary and/or non-proprietary device, may be an employee of a health care provider, health care facility, or health care insurance company. The radio user may transmit electronic documents or other information to the exemplary computer device 114 or a user thereof. The user of the exemplary computer device 114, or the exemplary computer device 114 alone and/or autonomously, may then transmit the electronic documents or other information to the authorized computer system 100.

As shown in FIG. 1A, exemplary computer devices 116 and 118 are coupled with a local area network (“LAN”) 124 which may be configured in one or more of the well-known LAN topologies, e.g. star, daisy chain, etc., and may use a variety of different protocols, such as Ethernet, TCP/IP, etc. The exemplary computer devices 116 and 118 may communicate with each other and with other computer and other devices which are coupled with the LAN 124. Computer and other devices may be coupled with the LAN 124 via twisted pair wires, coaxial cable, fiber optics or other wired or wireless media. As shown in FIG. 1A, an exemplary wireless personal digital assistant device (“PDA”) 122, such as a mobile telephone, tablet based compute device, or other wireless device, may communicate with the LAN 124 and/or the Internet 126 via radio waves, such as via Wi-Fi, Bluetooth and/or a cellular telephone based data communications protocol. PDA 122 may also communicate with the authorized computer system 100 via a conventional wireless hub 128.

FIG. 1A also shows the LAN 124 coupled with a wide area network (“WAN”) 126 which may be comprised of one or more public or private wired or wireless networks. In one embodiment, the WAN 126 includes the Internet 126. The LAN 124 may include a router to connect LAN 124 to the Internet 126. Exemplary computer device 120 is shown coupled directly to the Internet 126, such as via a modem, DSL line, satellite dish or any other device for connecting a computer device to the Internet 126 via a service provider therefore as is known. LAN 124 and/or WAN 126 may be the same as the network 420 shown in FIG. 4 and described below with respect thereto. One skilled in the art will appreciate that numerous additional computers and systems may be coupled to the authorized computer system 100.

The operations of computer devices and systems shown in FIG. 1A may be controlled by computer-executable instructions stored on a non-transitory computer-readable medium. For example, the exemplary computer device 116 may include computer-executable instructions for receiving electronic documents from a user and transmitting that information to the authorized computer system 100. In another example, the exemplary computer device 118 may include computer-executable instructions for providing electronic messages to the authorized computer system 100 and/or receiving electronic documents or other messages from the authorized computer system 100 and displaying that information to a user.

Of course, numerous additional servers, computers, handheld devices, personal digital assistants, telephones and other devices may also be connected to the authorized computer system 100. Moreover, one skilled in the art will appreciate that the topology shown in FIG. 1A is merely an example and that the components shown in FIG. 1A may include other components not shown and be connected by numerous alternative topologies.

FIG. 1B depicts a block diagram of an annotation system 140 according to one embodiment, which in an exemplary implementation, is implemented as part of the authorized computer system 100 described above.

FIG. 1B shows a system 200 for annotating the dataset 104 of the network-connected authorized computer system 100 shown in FIG. 1A. The system 200 may communicate with the authorized computer system 100 via a network 208, which may be the network 420 described below or network 124 or 126 described above. The system 200 may be separate and distinct from the authorized computer system 100, as described above. In another embodiment, the system 200 may be incorporated as an individual module within the authorized computer system 100. The system 200 may involve functionality to access, identify, select, annotate, accumulate, organize and/or otherwise manipulate electronic documents or messages that have previously been received and/or processed by the authorized computer system 100. The system 200 may involve functionality to supply, inject, receive, and/or otherwise communicate the electronic documents or messages to the authorized computer system 100 in a manner that mimics or mirrors the provision of electronic documents or messages from users using any of the previously described workstations and/or interfaces 116, 118, 122, 120, 114. As such, the authorized computer system 100 may accept and/or otherwise receive the synthesized electronic documents or messages from the system 200, and process or send them similar to how the authorized computer system 100 processes and sends other electronic documents or messages received from other sources. This will mimic the actual operation of the authorized computer system 100, but with controlled and/or specified data. It will be appreciated that the disclosed embodiments may be applicable to other types of electronic documents and messages, and authorized computer systems, beyond those described specifically with respect to the authorized computer system 100. Further, the dataset 104 or other datasets, and/or the data contained therein, may be communicated throughout the system using one or more data packets, datagrams or other collection of data formatted, arranged configured and/or packaged in a particular one or more protocols, e.g. FTP, UDP, TCP/IP, Ethernet, etc., suitable for transmission via a network 214 as was described, such as the dataset communication format and/or protocols.

The system 200 includes a processor 150 and a non-transitory memory 160 coupled therewith which may be implemented as processor 402 and memory 404 as described below with respect to FIG. 4. The system 200 may be an annotation system 140, as described above with respect to FIG. 1A. The system 200 further may include a dataset store 167, or database, configured to store one or more datasets involving a collection of data, or data items, received and/or processed by the authorized computer system 100. The data items may be organized in an ordered or standardized manner, such as including data indicating the type and corresponding values of data items that were received by the authorized computer system 100. As shown, the system 200 includes various logical functions, individual devices, and/or combined devices. The logical functions, individual devices, and/or combined devices may share the processor 150 as shown, or may include individual processors, as well as any combination or shared processing abilities over multiple processors. As such, multiple processors 150 may be used in dedicated applications for the particular individual devices, and/or combined devices, or in any shared combination.

The system 200 may include a data preparer 162 that is stored in the memory 160 and executable by the processor 150 to access the database 102 and the dataset 104 from the authorized computer system 100. The processor 150 may include circuitry or a module or an application specific controller as a means for accessing data from the database 102 and/or the dataset 104 from the authorized computer system 100, e.g. data items stored in the database 102. Each data item, or data record, of the database 102 of the authorized computer system 100 is stored in association with one or more properties, each property being associated with a corresponding value thereof.

The data preparer 162 may also be executable by the processor 150 to create summary information about the database 102. In one example, the data preparer 162 may assign a type to each property associated with a data item. This may only be necessary if each property type is not already available. In most cases, it is not necessary for most RDBMS systems because a type may already be assigned to each text column in the database 102. However, for semantic web ontologies, NoSQL databases (non-relational databases), or if text columns are used to represent data types other than strings (i.e. floating-point numbers, integers), then assigning property types may be necessary. Type assignments may be needed for computing prior probabilities for tags as described further below. The data preparer 162 may detect a type by a successive regular expression match in a reverse order of an entailment order. For example, numbers may be represented as strings, so string representations entail number representations. This is because numbers may be represented as a series of ASCII characters or numerically. If the regular expression matches all values for the property, the data preparer 162 may assign a type. Specifically, in one example, the data preparer 162 may match values according to regular expressions in the following order: integers, then floating-point numbers, then lower case text, then upper case text, then alpha characters, and then alpha-numeric characters. Examples of regular expressions for these categories may be the following: for integers, \hat{ }[+−]?[0-9]+$; for floating point numbers, \hat{ }[+−]?([0-9]*\\.)?[0-9]+$; for lower case text, \hat{ }[a-z]+$; for upper case text, \hat{ }[A-Z]+$; for alpha characters, \hat{ }[A-Za-z]+$; and for alpha-numeric characters, \hat{ }[A-Za-z0-9]+$.

In another example of creating summary information about the database 102, the data preparer 162 may calculate how much weight to give properties of an item in the database 102. This weight may act as a hyperparameter. As used herein, a hyperparameter is a customizable setting used to tune an algorithm's performance. This may be any setting that remains constant during both training and testing of a machine learning algorithm whose purpose is to increase better performance (i.e., like a knob for tweaking the output of a machine). In one exemplary embodiment, the data preparer 162 may use a value's (i.e., a string to indicate a name, or the integer “9” for an age) distribution over each property. In this way, the likelihood of a rare value belonging to a property when observing items (i.e., observing item nodes of a Bayesian network) is increased. This weight distribution method and set of heuristics are dynamic and easily modified a priori to the execution of the disclosed embodiments. Using this method, the probability may be defined as the number of occurrences of a value subtracted from the counts of all other values for a given property. More specifically, for some value d that belongs to property ψ, where d ∈ ψ, then the probability of this relationship is defined as:

$$P ( d ∈ ψ ) {=}^{△} \frac{\vert ψ \vert - \vert d ∈ ψ \vert}{\vert ψ \vert} \qquad ( 1 )$$

Given that floating point representation of numbers may be in the database 102 and may differ given a programming language's representation, other methods may be needed to calculate the distribution. Examples include kernel density estimation as a probability distribution function and persisted for fast calculation of values at run time.

The system 200 may include a tokenizer 164 that may be implemented as a separate component or as one or more logic components, e.g. first logic, such as on an FPGA that may include a memory 160 or reconfigurable component to store logic and a processing component to execute the stored logic, or as computer program logic, stored in the memory 160, or other non-transitory computer readable medium, and executable by the processor 150, such as the processor 402 and memory 404 described below with respect to FIG. 4, to cause the processor 150 to, or otherwise be operative to, segment the text of the dataset 104 into tokens, or strings of one or more characters, such as words or symbols, usually delimited by white space. The tokenizer 164 may be coupled with the data preparer 162. Segmenting, or parsing, the stream of text of the dataset 104 into words or symbols may be referred to as “tokenizing.” In one example, words, when tokenized, may be split on contractions and punctuations. The processor 150 may include circuitry or a module or an application specific controller as a means for segmenting or tokenizing the text of the dataset 104. The tokenizer 164 may be a software component, such as any now known or later developed data parser, that takes input data (frequently text) and builds a data structure, such as a parse tree, abstract syntax tree or other hierarchical structure, giving a structural representation of the input while checking for correct syntax.

It is noted that in one embodiment, the steps discussed above of the data preparer 162 creating summary information about the database 102 and the tokenizer 164 segmenting the text of the dataset 104 into tokens happens prior to annotating the text of the dataset 104 and if any data changes in the database 102 or the dataset 104, these steps must be repeated.

The system 200 may include a data analyzer 166 that may be implemented as a separate component or as one or more logic components, e.g. first logic, such as on an FPGA that may include a memory 160 or reconfigurable component to store logic and a processing component to execute the stored logic, or as computer program logic, stored in the memory 160, or other non-transitory computer readable medium, and executable by the processor 150, such as the processor 402 and memory 404 described below with respect to FIG. 4, to cause the processor 150 to, or otherwise be operative to, analyze the results of the tokenizer 164 tokenizing the dataset 104 of the authorized computer system 100 to identify tokens in the dataset 104 that match property values in the database 102 for predetermined database properties and determine whether the identified tokens in the dataset 104 represent values associated with a property in the database 102. The data analyzer 166 may be coupled with the tokenizer 164. The processor 150 may include circuitry or a module or an application specific controller as a means for identifying tokens in the dataset 104 that match property values in the database 102 for predetermined database properties and a means for determining whether the identified tokens in the dataset 104 represent values associated with a property in the database 102. As will be described in more detail below with reference to FIG. 5, examples of database properties and values may include first name, last name, age, and gender, and “Jack,” “Green,” “32,” and “M,” respectively. In one example, database properties may be predetermined based on the type of properties, and corresponding values, to be annotated. For instance, in the example mentioned above for annotating PHI, which will be discussed in greater detail below, the predetermined database properties may correspond to any or all properties that are considered to be PHI, such as names, social security numbers, driver's license numbers, birth dates, gender, ethnicity, diagnoses, treatment information, medical records and test results, prescription information, a preferred or emergency contact method, contact information for the preferred or emergency contact method, types of insurance, codes indicating health conditions, codes indicating procedures provided by a health care provider, types of benefits covered, costs of procedures provided by a health care provider, dates of service (i.e., when the procedures were performed by the health care provider), details regarding payment for health care, etc. The foregoing list is not exhaustive, and it is recognized that other types of properties may be considered PHI.

In one example, the data analyzer 166 may utilize a string searching algorithm, such as the Aho-Corasick algorithm, to identify candidate tokens in the dataset 104 that match property values in the database 102, since looking up all possibilities becomes computationally intractable. The data analyzer 166 may use the string searching algorithm on a document of the dataset 104 by character-level finite state automatons to find candidate spans of characters, or tokens, which can be used to find and match candidates. The string searching algorithm is given all values of all data from the database 102 using property identifiers, then persisted to disk. Using a known string searching algorithm such as the Aho-Corasick algorithm, it is to be understood that a finite state automaton is built from the data. An automaton table is then used in much the same way a regular expression state transition table matches text. It is advantageous that the algorithm may match all instances of a string of characters. For example, if the word “can” and “scanned” are given (i.e., input values of the algorithm), the algorithm will find both the complete first word “can” and in the latter text substring “can” in “s(can)ned”. In this regard, the string searching algorithm for building the automaton and matching may be very performant. Experiments show that candidate matching for each document may only take a fraction of a second. In exemplary embodiments, only matches on word boundaries may be matched, as provided by the tokenizing step discussed above. If a string of characters (i.e., token) is not found in the database 102, the string/token is not a candidate and the process ends for that string/token.

Once candidate tokens are found, the token data may be linked to a property value in the database 102. However, the possibility of whether or not the candidate token originates from the database 102 must first be considered. Even if the candidate token is found in the database 102, the candidate token still might not have come from the database 102. Therefore, it must be determined whether the candidate token originated from, or was derived from, the database 102. This may be performed by determining whether or not tokens actually represent property values in the database 102, as opposed to the tokens representing something not included in the database 102.

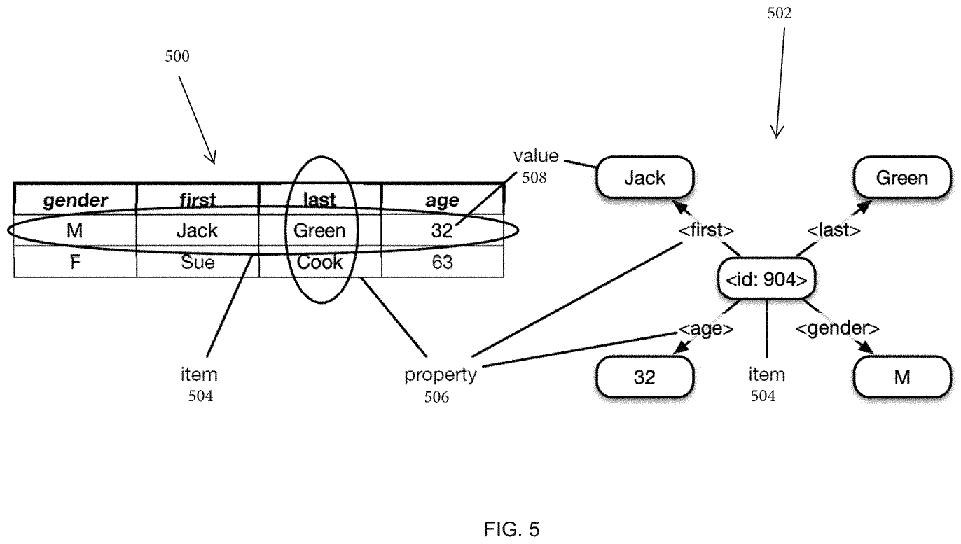

In this regard, the data analyzer 166 may also cause the processor 150 to determine whether the identified tokens in the dataset 104 represent values associated with a property in the database 102. By determining whether the identified tokens in the dataset 104 represent values associated with a property in the database 102, the data analyzer 166 is determining whether data from the dataset 104 is derived from, or originated from, the database 102. As noted above, some data appearing in the dataset 104, or unannotated corpus, might match data contained in the database 102 despite not having been derived from the database 102. Annotating such data may lead to false positives and decrease the accuracy of the proposed annotation system 140. Since the annotation system 140 may not know for sure how such data got into the dataset 104, the annotation system 140, by way of the data analyzer 166, analyzes the probabilities that the data of the dataset 104 (i.e., identified tokens) came from the database 102 to know if the database property for that data applies. In the example mentioned above where the document processing module 106 generates a document that mentions a person's name and number of children, even though the specific values of certain data items, such as the person's name and number of children in the current example, may match data values in the database 102, the values of these data items may not have originated in the database 102. For example, if the email in this example mentioned that a particular patient has 6 children, the tokenizer 164 may segment the text of this email into tokens, where the number “6” is one of the tokens. The data analyzer 166 may then identify the token “6” as matching a property value in the database 102 using, for example, a string searching algorithm as discussed above. In this example, the data analyzer 166 may match the token “6” to a value in the database 102 associated with the property “age,” where age is one of the predetermined database properties, because one data item in the database 102 has a corresponding value of “6” for the data item property of “age” (i.e., a patient represented by a data item in the database 102 is 6 years old). In this example, even though the token “6” matches a property value of “6” in the database 102, the token “6” does not represent a value of the “age” property. Rather, the token “6” represents a particular number of children. Thus, since the token “6” in this example does not represent a value associated with a property in the database 102, the token “6” will not be annotated, thus avoiding a false positive annotation. For example, if the proposed annotation system 140 is used to annotate PHI in text, the token “6” will not be annotated since the value of “6” represents a particular number of children (not PHI) rather than age (PHI). This example is further described below with respect to FIG. 6.

To determine whether data from the dataset 104 derived from the database 102, the data analyzer 166 of the proposed annotation system 140 may use a known language model along with a Bayesian network to calculate probabilities of whether the identified tokens originated from the database 102. Specifically, the known language model may be used to compute prior probabilities of whether the identified tokens represent database property values and the Bayesian network may be used to compute posterior probabilities of whether the identified tokens represent database property values based on the prior probabilities. Determining whether a respective identified token represents a value associated with a property in the database is then based on the calculated posterior probability. In another embodiment, the data preparer 162 may perform this calculation after creating summary information about the database 102.

Computing prior probabilities is helpful for determining whether a candidate token is derived from the database 102 and for assigning a confidence probability for each candidate token. The calculated prior probabilities will then be used by the Bayesian network for calculating posterior probabilities. For example, observing nodes in the Bayesian network may start with a node having the highest calculated prior probability, and different iterations of observing nodes may also be tied to the calculated prior probabilities. In one embodiment, the known language model used to compute prior probabilities is the Google Ngram data set (for more information on this dataset, see https://storage.googleapis.com/books/ngrams/books/datasetsv2.html). The Google Ngram data set contains a list of scanned words originating from books dating back to the 19th century by Google. Each entry in the data set is an n-gram, which is a string of words with a fixed length. The n in n-grams is the length, such as a bi-gram, which has an n-gram of n=2. In the vast majority of cases, any PHI that is found in the exemplary dataset 104, such as, for example, doctor's notes, would not be found in any book scanned by Google. Therefore, it is assumed that the Google Ngram data set and the exemplary dataset 104 of the disclosed system are mutually exclusive data sets. By this assumption, n-grams are used to calculate the prior probability for each token found in the dataset 104 thereby proving a reasonable probability estimate of the null hypothesis (the hypothesis that the text is drawn from the scanned books) or from text containing PHI that originates from the database 102. The term “null hypothesis” refers to the probability that there is no special order to a phenomenon. The specific null hypothesis for the disclosed embodiments may be that the tokens found in the database 102 do not originate from the database 102. Thus, calculating a prior probability of an identified token is based on not only whether a token is found in the Google Ngram data set, but the number of occurrences (i.e., a prevalence) of the identified token in the Google Ngram data set.

The data analyzer 166 may calculate the prior, n-gram probabilities using tri-grams, which is an n-gram with the length of three. To calculate the prior probability, the data analyzer 166 may use a statistical equation. For example, let count (wᵢ₋₂, wᵢ₋₁, wᵢ) be the number of occurrences of the tri-gram in the n-gram data set using the token t as the third word wᵢ. The hyperparameter “top i n-gram” (Nᵢ) is the top i^{th} ranked tri-gram count. In this example, the prior probability of an identified token may be defined as:

$${P}_{ng} ( {w}_{i} ∈ 𝒟 \text{\textbar} {w}_{i - 2} , {w}_{i - 1} ) {=}^{△} \frac{count ( {w}_{i - 2} , {w}_{i - 1} , {w}_{i} )}{{N}_{i}} \qquad ( 2 )$$

where [symbol] is the database 102 and P_{ng} (wᵢ∈ [symbol] ) is the n-gram based probability for token wᵢ, which originates from the database 102. In other words, this expression states that the prior probability that the token belongs to the database 102 given the previous two words is defined as the number of occurrences of the tri-gram in the n-gram data set, where the token is the third word, divided by the top i^{th} ranked tri-gram count hyperparameter. The choice for this parameter in place of using N₀ is that the first several entries of the n-gram data set are punctuation only. Also, tri-gram distribution roughly follows Zipf's law as expected. A general rule states that any word in a natural language has a frequency of about one half that of the next most frequent word. Other statistical methods may be used such as conditioning on the Part of Speech (POS) tags or features from a Semantic Role Labeler (SRL). Neural network methods such a deep network with word embeddings may also provide a language model that provides next-word probability distribution given some token window.

This exemplary formulation considers the relative probability of tri-grams, which is why it is used successfully for many natural language processing (NLP) tasks as language models and word prediction. However, this approach at times may yield poor results for out of domain data, which for this disclosed process, means applying estimates from the scanned books of the Google Ngram data set and to, for example, doctor's notes. Two issues with using the Google Ngram data set for estimating the null hypothesis for data inclusion given the out of domain issue includes missing “out of vocabulary” tri-grams from the database 102 and the scale of the data. To account for this, the data analyzer 166 may take into account both n-gram misses and scaling, which are described below.

For missing tri-grams, the data analyzer 166 can search for uni-grams (an n-gram length of one) and bi-grams (an n-gram length of two) with “like where clause” expressions in the n-gram database. The n-gram data set may be added to a RDBMS as a processing step to efficiently find the counts for each trigram. This count may need to be scaled to account for higher occurrences proportionate to the number of n-grams found. That is, the data analyzer 166 may first search bi-grams and finally search for single token uni-grams. The traditionally smoothing constant used in a Katz back-off-model may not be used since the goal is to compute token prior probabilities rather than train a language model. In many cases the proposed method may not get results even for uni-grams like social security numbers and other unique identifiers. In these cases, the prior probability may be defined as the number of combinations for a string based on the property type assigned as described above. For example, if a nine-digit number is found that might be a social security number, then the perplexity (a measure of the probability of a word in the dataset 104) of this token w, and generally speaking the odds of finding this particular string, is:

$$PP ( w ) = \frac{1}{{10}^{9}} , \qquad ( 3 )$$

assuming each digit is equally likely to happen. More generally, the probability of the token missing from the n-gram database (Pₘ) may be estimated as:

$${P}_{m} {=}^{△} ( w ) = \frac{1}{{c}^{slen ( w ) \cdot d}} , \qquad ( 4 )$$

where c is the number of combinations of a single character in token w given by the data type (i.e., float vs string) and s len (s) is a function that returns the number or characters of string s. The hyperparameter dampen (dₛ) may be used to slow the exponential growth on long strings. In one example, a (dₛ) value of 0.4 may be used. Other (dₛ) values may be used as well, such as 0.38 and 0.44.

The perplexity, or the corpus branching factor (i.e., probability distribution over a corpus of the likelihood of any word), of any domain-specific corpus may be much smaller than that of a data set like that of Google's n-grams, and thus, the token prior probability estimates may be too low. This may be because the language expressed in certain datasets, such as, for example, doctor's notes, may be more limited than that of the scanned books. Heuristically scaling with a hyperparameter may be used to ameliorate these low estimate issues. The null hypothesis scale (r) hyperparameter scales the null hypothesis higher, with exemplary values ranging from 1-20. Preferably, a value of 4 may yield the best overall performance. The linear scaling may help, but it may not be enough since the logarithmic term decay that follows Zipf's distribution may be too steep. For this reason, a scaled softmax is calculated on [1-P(w), P(w)], which exponentiates the n-gram distribution. This scaling un-smooths the function to “tighten” or “sandwich” the distribution and minimize the spread. The hyperparameter n-gram compression (sᵤ) may be used to compresses the distribution. In one example, a value of 1.5 may be used for (sᵤ). Other values are possible. This softmax scaled function is defined as:

$$σ ( X , {s}_{u} ) = \frac{exp ( X \cdot {s}_{u} )}{1 + exp ( X \cdot {s}_{u} )} . \qquad ( 5 )$$

The final formulation of the prior probabilities that incorporates the missing n-gram and domain perplexity in equations 2, 4, and 5 is given below:

$$\begin{matrix}{P}^{′} ( {w}_{i} ∈ 𝒟 \text{\textbar} {w}_{i - 2} , {w}_{i - 1} ) {=}^{△} \{ \begin{matrix}{P}_{ng} ( {w}_{i} ∈ 𝒟 \text{\textbar} {w}_{i - 2} , {w}_{i - 1} ) & if {w}_{i - 2} , {w}_{i - 1} , {w}_{i} ∈ {𝒟}_{ng} \\ {P}_{m} ( {w}_{i} ) , & otherwise\end{matrix} & ( 6 ) \\ {P}_{sc} {=}^{△} {P}^{′} ( {w}_{i} ∈ 𝒟 \text{\textbar} {w}_{1 - 2} , {w}_{i - 1} ) \cdot r & ( 7 ) \\ {P}_{t} ( {w}_{i} ∈ 𝒟 \text{\textbar} {w}_{i - 2} , {w}_{i - 1} , s ) {=}^{△} {σ}_{s} ( ( 1 - {P}_{sc} , {P}_{sc} ) , s ) , & ( 8 )\end{matrix}$$

where [symbol] _{ng} is the n-gram database, P_{sc} is the scaled probability, and Pₜ given in equation 8 is the prior probability for a given candidate token, which is the belief that the candidate token originated from the database 102. The null hypothesis is the belief the candidate token appears in the database 102 by chance, and did not originate from it is equal to 1-Pₜ.

Once the prior probabilities for each identified token are calculated, the data analyzer 166 may be executable by the processor 150 to calculate posterior probabilities for each identified token using a Bayesian network. The construction of the Bayesian network will be discussed in more detail below. In another embodiment, the data preparer 162 may perform this calculation after creating summary information about the database 102. In general, posterior probability is the probability an event will happen after additional evidence or background information has been considered or known. The prior probability is some observed probability of an event that is considered a priori and assumed. On the other hand, the posterior probability is the probability an event will happen after taking into account new information. This “new information” is the probably of an occurrence of an event. The posterior probability is calculated using Bayes's theorem as a function of the prior probability, which is defined as P(AIB)=P(BIA)*P(A)/P(B), where P(A) is the prior (initial belief), P(AIB) is the posterior (the new belief) taking into consideration (conditioning on) event B. This posterior distribution is a way to estimate an outcome for some event for which we cannot or do not want to sample by trial, (the frequentist approach). In other words, the posterior distribution estimates the probably of an event after the data of an initial state of an event has been observed. Thus, similar to the prior probability discussed above, the posterior probability also represents the probability that an identified token represents a value associated with a property in the database 102. The posterior probability of the token is initially calculated as the posterior probability of the Bayesian graph node and later by observing nodes in the Bayesian network (i.e., the new information being taken into account, or the likelihood of something happening).

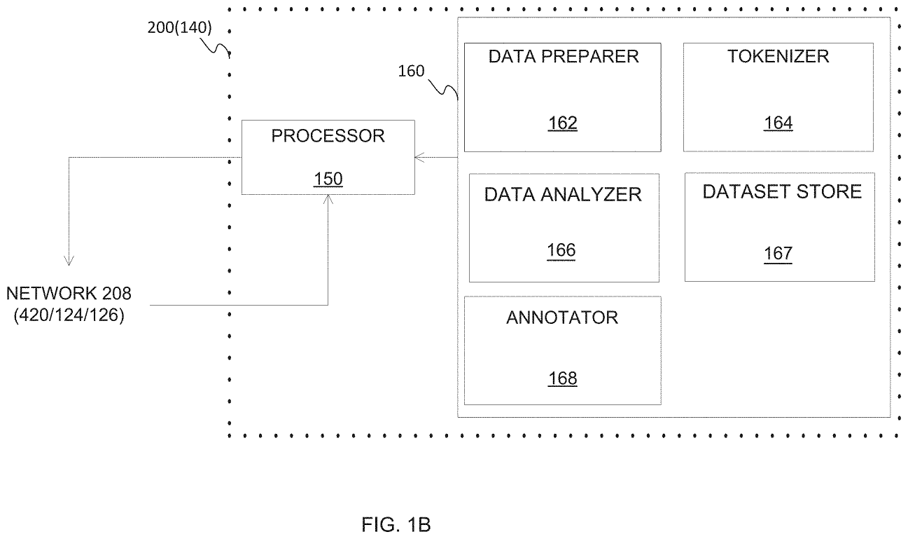

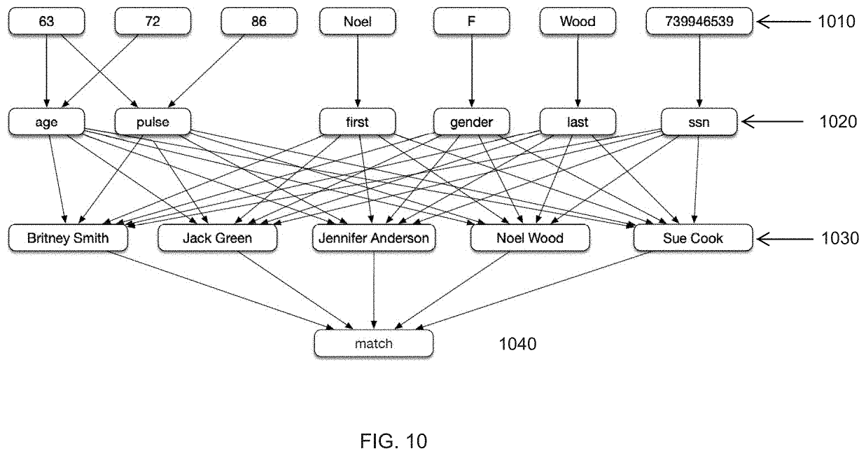

As shown in FIG. 2, the proposed method of calculating posterior probabilities is iterative in that the data analyzer 166 may calculate posterior probabilities for each identified token starting with observing a bottommost child node of the Bayesian network having the highest calculated prior probability (maximum a posterior likelihood), where, once observed, the prior probabilities of the remaining, unobserved nodes are adjusted or refined accordingly and filtered based on predetermined probability thresholds. This process may be referred to as a Bayesian inferencing process. The bottommost child node of the Bayesian network may be referred to as the match node (i.e., a network node that ties all items together), which will be discussed in further detail below with reference to FIG. 10 and the construction of the Bayesian network. The data analyzer 166 may then move on to parent nodes and observe them. As shown in FIG. 2, this process repeats for each layer of parent nodes of the child node in the Bayesian network (i.e., the item nodes and property nodes) and ends when there are no more parent nodes to observe. As discussed above, observing a node is a process where one state of a random variable, and thus one row in a CPT, is given a probability of 1 (100%) and all other states of that variable is given a 0 (0%). Therefore, to observe nodes, the data analyzer 166 may be executable by the processor 150 to maximize the probability of a state on a Bayesian network node. In one embodiment, to maximize the probability of a state of a node, the data analyzer 166 may be executable by the processor 150 to change the state of an observed node to 1, or 100% probability of that state occurring.

The data analyzer 166 may also be executable by the processor 150 to determine whether a respective identified token represents a value associated with a property in the database 102. The data analyzer 166 may make this determination based on the calculated posterior probability for an uppermost parent node representing the respective identified token.

A Bayesian network is constructed using the matched tokens identified above and the data that references the values of those tokens. The data analyzer 166 may be executable by the processor 150 to construct the Bayesian network. In another embodiment, the data preparer 162 may construct the Bayesian network after creating summary information about the database 102. In the Bayesian network, each token, property, and item are represented as nodes in a graph. A Bayesian network has a CPT for each node, the CPT being defined based on the relationship given in the database 102. A high level overview of the construction of the Bayesian graph includes: a) tokens are removed (as discussed below) and those left are grouped by value, b) properties that have values for the remaining tokens are added, c) items that have token values for respective properties are added, and d) the match node (node that ties all items together) is added. Note that the structure of the Bayesian network reflects the order of adding and connecting nodes. That is, token nodes are the highest-level, or uppermost, parent nodes. The token node's children are the property nodes, whose children are the item nodes, all of which have the single match node as their child. Token nodes that have values for properties are connected to those respective property nodes.

Once candidate tokens are identified as matching property values in the database 102, as discussed above, each token is treated as a separate instance with its own text span, offset, and hypothesis prior probabilities. Tokens are discarded if their prior probabilities are not equal or greater than the predetermined token prior probability threshold (referred to as Kₚ). This step may be necessary to put some limits on the spatial growth of the CPTs, which may have an impact on the time complexity of the Bayesian network inferencing algorithm. After candidate tokens not meeting the predefined threshold are removed, those that are left over are grouped by value in a token node (a node that represents a token value in the Bayesian network) and added to the Bayesian network graph as nodes. The CPT of each token node is taken from the computation of prior probabilities as described above.

Next, to add the property nodes, the database 102 is used to query those properties and items that match the identified tokens. This query may also include the distribution data points calculated in the processing step described above. Only those properties for tokens that connect to items are added to the graph. The CPT for each property is based on the parent binary token node variables with the tag node posterior probabilities over those parent tokens and the null hypothesis state. As mentioned above, a token node represents a unique string token with zero or more occurrences in a document of the dataset 104. Let the number of the i^{th} token in the document vocabulary t ∈V that repeats n times be tᵢ=n. This token count yields the probability estimation for the property CPTs. Also, as discussed above, the probability distribution of some database value d belonging to property ψ is P(d ∈ ψ) (see Equation 1 above). The formula given for the computation of the property CPTs is the hyperparameter property strategy (αₛ) with two settings: the number of tokens (\tilde{P}ₐ), based on the number of tags and as a function of the distribution calculated as described above (\tilde{P}_{d}). A hyperparameter token duplication rate (Sₜ) may be used in the distribution function as:

\tilde{P}_{d}[1+((tᵢ−1)·sₜ)]·P(d ∈ ψ), (9)

where d is the value in the database 102 and ψ is the property node that contains the CPT being populated. In one embodiment, the token duplication rate (sₜ) may be a value of 2.0. Other values are possible.

The notation [symbol] (ψ) may be used to identify the parents of property ψ, which are the token nodes. Now let the score proportionate to the probability in the cell in row i of column j of the CPT be:

$$\tilde{{P}_{i , j}} = \{ \begin{matrix}{\tilde{P}}_{a ,} & if {α}_{s} = all , \\ {\tilde{P}}_{b} , & if {α}_{s} = distribution ,\end{matrix} \qquad ( 10 )$$

where i ∈ [0, [symbol] ] since all tokens are binary random variables and j ∈ [0, | [symbol] (ψ)|]. The dimension of the CPT is [symbol] ×| [symbol] (ψ)|+1 since the null hypothesis is added as an additional column. Each row of the CPT is a probability distribution over the corresponding token parents coming from the database 102 with an additional column representing that combination's null hypothesis and is calculated as follows:

$${\tilde{P}}_{{h}_{i}} = {max}_{j} \{ 0 , 1 - \tilde{{P}_{i , j}} \} , \qquad ( 11 )$$

where \tilde{P}ₕ_{ᵢ} is the null hypothesis probability of the i^{th} row of the CPT. The scores calculated as such are proportional to the probability per row, and therefore, are normalized across columns. Very loosely, the probability “spills over” to the null hypothesis. This may happen when there is insufficient potential for combinations, such as when all parents have the “out of database” state, in which case the null hypothesis is maximized and there is 100% chance the value d is not in the database 102.

Next, after the property nodes are created, their children nodes (i.e., item nodes) are created as the next level deeper in the Bayesian graph. The property and item nodes are connected if there is at least one token found in the database for the connected item node. For RDBMS type databases, this implies the two layers are fully connected unless there is special treatment for null values. For semantic web and NoSQL databases, avoiding connections helps the computation complexity during inferencing. Each item node represents a binary random variable, each having a CPT parameterized by states of the parent property nodes, and are the Cartesian product of those properties. Let the parents of item ξ be [symbol] (ξ)=Ψ, then the probability is drawn from the computed distribution, as described above. However, the distribution may be “sliced” by adding ξ to the criteria when computing each data point. Each row in the CPT of the ξ node has two columns that indicate if the item from a document of the dataset 104 originates form the database 102 with states: present or absent. Each row is a combination of parent property nodes Ψ with states denoted as s₁₀₄ ∈ X_{ψ} where ψ ∈ Ψ and s_{ψ} is a state that belongs to the property's random variable X_{ψ}. Furthermore, let the property nodes Ψ that match states s_{ψ} be Ψₘ (where m denotes a match), let P(d ∈ ψ, d=s_{ψ})∀ s_{ψ} ∈ Ψₘ be the probability distribution for value d, property ψ and random variable state s_{ψ}, let [symbol] (⋅) be the indicator function, and let the hyperparameter item strategy (βₛ) specify a CPT cell calculation. From the graph structure between the property and item levels and binary nature of the item node as a random variable, it may be clear that the number of rows for each node's CPT is 1+Σ_{ψ ∈ Ψ} | [symbol] (ψ)| since the number of states s_{ψ} ∈ X_{ψ}, as defined above, is the number of tokens for each property including the null hypothesis state. Therefore, the dimension of the item CPT is Σ_{ψ ∈ Ψ} |X_{ψ}|×2. Now let the probability that the item is in the database 102 for the i^{th} row of the ξ node's CPT be:

$${P}_{i} = \{ \begin{matrix}1 ( \vert {Ψ}_{m} \vert > 0 ) , & if {β}_{s} = all \\ min ( 1 , 1 - {\sum }_{ψ ∈ Ψ} {\sum }_{d ∈ ψ} P ( d ∈ ψ , d = {s}_{ψ} ) ) , & if {β}_{s} = distribution \\ \frac{\vert {Ψ}_{m} \vert}{Ψ} , & if {β}_{s} = matched .\end{matrix} \qquad ( 12 )$$

From Pᵢ, the probability of item ξ not being in the database may be calculated as 1−Pᵢ.

The final step in construction the Bayesian network is to add the match node. The match node is the final singular child node of all others in the Bayesian network. It represents the best match as a distribution across all items for the matched tokens found in the document given its direct item parent nodes, as discussed in the preceding section above. Similarly to the property nodes, the data analyzer 166 may create the CPT with each row as a combination of parent item nodes Ξ with states denoted as s_{μ} ∈ X_{μ} where μ ∈ Ξ and s_{μ} are states that belong to an item's random variable X_{μ}. Since each parent is a binary random variable, the dimension of the CPT is | [symbol] (μ)|× [symbol] where μ is the match node. Each entry of the match node has a proportionate contribution based on the parent's item state database membership, with the exception of the null hypothesis state, which is 1 for all items not in the database 102. More specifically, let Ξ be the set of item nodes with ξ ∈ Ξ, which are the parents of match node μ, and let the item nodes Ξ that match states s_{μ} be Ξₘ, where m is used to denote an item match. Then the probability of the i^{th} row of the match node CPT is:

$${P}_{i} = \{ \begin{matrix}0 & if \vert {Ξ}_{m} \vert = 0 ⩓ {s}_{μ} \neq null hypothesis \\ 1 & if \vert {Ξ}_{m} \vert = 0 ⩓ {s}_{μ} = null hypothesis \\ \frac{\vert Ξ \vert}{\vert {Ξ}_{m} \vert} & otherwise .\end{matrix} \qquad ( 13 )$$

Once tokens in the dataset 104 are identified and determined to represent values associated with database properties, the tokens may be annotated. Referring back to FIG. 1B, the system 200 may include an annotator 168 that may be implemented as a separate component or as one or more logic components, e.g. first logic, such as on an FPGA that may include a memory 160 or reconfigurable component to store logic and a processing component to execute the stored logic, or as computer program logic, stored in the memory 160, or other non-transitory computer readable medium, and executable by the processor 150, such as the processor 402 and memory 404 described below with respect to FIG. 4, to cause the processor 150 to, or otherwise be operative to, annotate the identified tokens of the dataset 104 when the identified tokens are determined to represent values associated with a property in the database 102. The annotator 168 may be coupled with the data analyzer 166. The processor 150 may include circuitry or a module or an application specific controller as a means for annotating the identified tokens of the dataset 104 when the identified tokens are determined to represent values associated with a property in the database 102. To carry out the annotations, the annotator 168 may be executable by the processor 150 to associate a tag with each identified token and assign annotation attributes for each tag. In this regard, an annotated token may be referred to as a tag. In one embodiment, the annotation attributes may include identification of database data items, database properties, database property values, a probability that the identified tokens represent values associated with a property in the database 102, a determination of whether the identified tokens represent values associated with a property in the database 102, character span information for characters of the identified tokens, an annotation identification, or combinations thereof. The foregoing list is not exhaustive, and the annotator 168 may assign other annotation attributes as well.

In computer text processing examples, the annotator 168 may use a markup language to perform the annotating. Markup languages, like XML and HTML, annotate text in a way that is syntactically distinguishable from that text. Markup languages can be used to add information about the desired visual presentation, or machine-readable semantic information, as in the semantic web. In Java programming language, annotations may be used as a type of syntactic metadata in the source code. Variables, parameters, methods, classes, and packages may be annotated. The annotations may be embedded in class files generated by a compiler and may be retained by the Java virtual machine, which may influence the run-time behavior of an application. It may also be possible to create meta-annotations out of the existing ones in Java.

The tags, i.e., the annotations (including annotation attributes such as those mentioned above) and associated tokens may be stored in a memory, such as memory 160, in a data structure together with the associated database properties and database values as an annotated dataset. Referring back to FIG. 1B, the system 200 may include a dataset store 167 that may be implemented as a separate component or as one or more logic components, e.g. first logic, such as on an FPGA that may include a memory 160 or reconfigurable component to store logic and a processing component to execute the stored logic, or as computer program logic, stored in the memory 160, or other non-transitory computer readable medium, and executable by the processor 150, such as the processor 402 and memory 404 described below with respect to FIG. 4, to cause the processor 150 to, or otherwise be operative to, store the annotations and associated database properties and database values in a memory as an annotated dataset. The processor 150 may include circuitry or a module or an application specific controller as a means for storing the annotations and associated database properties and database values in a memory as an annotated dataset. In another embodiment, the annotator 168 may be executable by the processor 150 to cause the annotations and associated database properties and database values to be stored in a memory, such as memory 106, as the annotated dataset. In another embodiment, the tags, annotations, annotation attributes, and associated database properties and values may be stored in a separate database, such as dataset store 167, as the annotated dataset. In one example, the annotations may be loaded into a relational database with the true labels and text stored separately.

The annotated dataset may then be used to train a machine learning model, where the result of the training is a machine learned model. In one example, the machine learning model is a machine learning network and the machine learned model is a machine learned network. In one example, the annotated dataset may be used to train automated, machine learned systems or models to identify text in another dataset using the machine learned model, such as identifying and tagging PHI in that other dataset. In another example, the annotated dataset may be used to train natural language processing systems to identify and tag PHI.

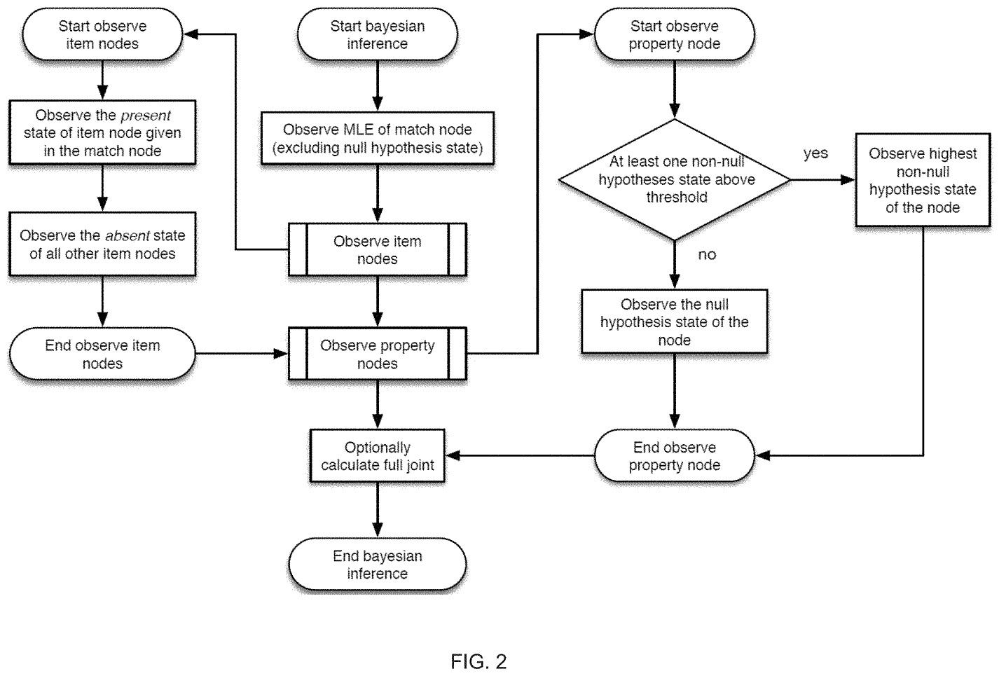

FIG. 3 depicts a flow chart showing operation of the annotation system 140 of FIGS. 1A and 1B. In particular, FIG. 3 shows a computer implemented method for automatically annotating a first dataset. The operation includes segmenting the text of the first dataset into tokens (Block 310), identifying tokens in the first dataset that match property values in the database for predetermined database properties (Block 320), determining whether the identified tokens derived from the database, i.e., whether the identified tokens in the first dataset represent values associated with a property in the database (Block 330), and annotating the identified tokens when the identified tokens are determined to be derived from the database (i.e., represent values associated with a property in the database) (Block 340), where the annotating includes associating a tag with each identified token and assigning annotation attributes for each tag. Additional, different, or fewer indicated acts may be provided. For example, storing the annotations and associated database properties and database values in a memory as an annotated dataset (Block 350) may be included. In another example, summary information about the database may be created (Block 315). The indicated acts may be performed in the order shown or other orders. The indicated acts, alone or in combination, may also be repeated, for example, determining whether the identified tokens derived from the database (Block 330), annotating the identified tokens when the identified tokens are determined to be derived from the database (Block 340), and storing the annotated dataset (Block 350) may be repeated. The indicated acts may also be performed automatically, either individually or as a whole, by the annotation system 140 as described above.

Prior to the text of the first dataset being segmented (Block 310), the database and the first dataset must be provided, or accessed. For example, the authorized computer system 100 may provide the database, such as database 102, and the first dataset, such as dataset 104. In another example, a user using any of the previously described workstations and/or interfaces 116, 118, 122, 120, 114 may access the database and/or the first dataset via the workstations and/or interfaces 116, 118, 122, 120, 114 of the authorized computer system 100 via wide area network 126 and/or local area network 124, the wireless hub 128, or the radio 132. The database and the first dataset may be provided in any form. In one example, the database and the first dataset may be provided in whole or in part. For example, only the most current data in the database and first dataset from the past year may be provided. In another example, the entire historical collection of data for both the database and the first dataset may be provided. As indicated above, after the database and first dataset are provided or accessed, if the underlying data in the database is changed, the proposed method will need to be restarted from the beginning.

As discussed above, the first dataset includes text, where a portion of the text contains data derived from the database. In one embodiment, the data derived from the database contains protected health information. Also as discussed above, the database includes a plurality of data items, each data item having one or more properties, each property of the one or more properties having an associated value. The database may also be structured with a pre-defined data model or format.

The first dataset may include a plurality of electronic documents relating to a plurality of patients. For example, the first dataset may include emails, reports, records, notes, or any combination thereof. In one example, the text of the first dataset is unstructured without a pre-defined data model or format. As discussed above, examples of unstructured data may include at least documents, journals, books, health records, metadata, audio, video, analog data, images, files, and unstructured text such as the body of an e-mail message, Web page, or word-processor document. In another example, the text of the first dataset is structured with a pre-defined data model or format. Structured data is data in a defined format, or code, that makes it easily readable and/or searchable by a computer. Examples of structured data includes at least JavaScript Object Notation (JSON) and Extensible Markup Language (XML) formatted files. In yet another example, a portion of the text of the first dataset is structured and another portion of the text of the first dataset is unstructured.

In one embodiment, the database and the first dataset are proprietary to an entity authorized under regulatory guidelines to possess the data in the database and the first dataset. As discussed above, examples of such authorized entities include at least healthcare providers, healthcare facilities, and health insurers. Regulatory guidelines imposing such requirements may include HIPPA and the GDPR.

The text of the dataset 104 of an authorized computer system 100 may be segmented (Block 310) using any technique. For example, the tokenizer 164 of the annotations system 140 may segment the text of the first dataset into tokens, or strings of one or more characters, such as words or symbols, usually delimited by white space. Any technologies, now known or later developed, such as those discussed above with respect to the tokenizer 164, may be used to segment the text of the first dataset into tokens. For example, the text of the first dataset may be segmented using any now known or later developed data parser. In another embodiment, the data preparer 162 may segment the text of the first dataset.

Tokens in the first dataset that match property values in the database for predetermined database properties may be identified (Block 320) using any technique. For example, the tokens in the first dataset may be identified by detecting tokens using a string searching algorithm. In an embodiment, the string searching algorithm may be the Aho-Corasick algorithm. The predetermined database properties may be properties associated with specific types of data. For example, the predetermined database properties may be database properties associated with healthcare related data. In this example, the healthcare related data may contain PHI, such as any information about health status, provision of health care, or payment for health care that can be linked to a specific individual, such as the examples listed above with respect to FIG. 1B.

Whether the identified tokens in the first dataset represent values associated with a property in the database (i.e., whether the tokens derived from, or originated from, the database) may be determined (Block 330) using any technique. In one embodiment, the determination is made using a second dataset, such as a known language model, and a Bayesian network. For example, the known language model may be constructed using n-grams using Google' s Ngram data set. In this example, the known language model may be used to calculate a prior probability, for each token identified in the previous step, of whether the identified token represents a value associated with a property in the database. In this example, the prior probability is calculated based on a prevalence of the identified token in a second dataset, such as, for example, Google's Ngram data set. In this example, the first dataset and the second dataset are mutually exclusive.

In this embodiment, determining whether the identified tokens in the first dataset represent values associated with a property in the database also includes iteratively calculating a posterior probability, for each identified token, of whether the identified token represents a value associated with a property in the database based on a Bayesian network. In this example, the iterative calculating starts with observing a bottommost child node of the Bayesian network having the highest prior probability calculated in the previous step and repeats for each layer of parent nodes of the child node of the Bayesian network. In an embodiment, the iterative calculation includes refining the prior probability calculated in the previous step. In this example, the refinement of the prior probability is based on observing nodes for each layer of parent nodes of the Bayesian network and filtering refined prior probabilities based on predetermined probability thresholds. In one example, observing nodes of the Bayesian network includes maximizing the probability of a state on a Bayesian network node. For example, the state of an observed node may be changed to 1 (i.e., probability of 100% of that state occurring). In this example, the states of all other unobserved nodes may be changed to 0 (i.e., probability of 0% of that state occurring). As discussed above, the state of a node may refer to the state of a random variable, and thus one row in a CPT.

In this embodiment, the determination step also includes determining whether a respective identified token represents a value associated with a property in the database based on a posterior probability for an uppermost parent node representing the respective identified token, which was calculated in the previous step. In this example, the uppermost parent node is a node at the highest-level of the Bayesian network that represents a token.

A challenge with conventional methods of annotating a dataset is that the annotating is performed manually, which is cumbersome, time consuming, prone to errors, and costly. Further, the amount of necessary annotated data needed in order to train conventional systems using that annotated data may not be available. The annotated dataset from using the disclosed embodiments disclosed herein does not need to be annotated manually, since the disclosed system is able to automatically annotate the dataset using proprietary information, such as the proprietary database and first dataset mentioned above. As mentioned above, this is a specific manner of automatically annotating a dataset, or corpus, using a known language model in conjunction with a Bayesian network, which provides a specific improvement over prior systems resulting in an improved data annotation system for creating an annotated corpus for data identification software systems.

The identified tokens of the first dataset may be annotated (Block 340) using any technique. In an embodiment, the identified tokens of the first dataset are only annotated when the identified tokens represent values associated with a property in the database, as determined in the previous step. In one example, annotating includes associating a tag with, or assigning a tag to, each identified token and assigning annotation attributes for each tag. In an embodiment, the tag reflects at least the probability that the annotated token was derived from the database and the database property thereof. For example, the tag may include data associations for a posterior probability calculated in the previous step and for a database property linked to the annotated token. In one example, the annotated token may be referred to as a tag. In one example, the annotation attributes include identification of database data items, database properties, database property values, a probability that the identified tokens represent values associated with a property in the database, a determination of whether the identified tokens represent values associated with a property in the database, character span information for characters of the identified tokens, or combinations thereof.

The annotations and associated database properties and database values may be stored in a memory as an annotated dataset (Block 350) using any technique. In an embodiment, the annotations and associated database properties and database values are stored in a data structure with associations. For example, the annotated dataset may be stored in a relational database. In this example, the relational database may include data associations, or links, between annotations attributes associated in the previous step, database properties, identified tokens, and corresponding values thereof. In one embodiment, the annotated dataset stored in this step may be used to train a machine learning model, such as, for example, a machine learning network. In this example, the result of the training is a machine learned algorithm, such as, for example, a machine learned network.

In an additional step, summary information about the database may be created (Block 315) using any technique. In an embodiment, creating the summary information for the database may include assigning a type of property associated with a data item in the database, as discussed above. In one example, a type of property may include integers, floating point numbers, upper-case text strings, and lower-case text strings. In another embodiment, creating the summary information for the database may include calculating how much weight to give properties of an item in the database, as discussed above. For example, the weight assigned to a property of an item in the database may be calculated using Equation (1) above. In another example, a kernel density estimation as a probability distribution function may be used.

Referring to FIG. 4, an illustrative embodiment of a specialized computer system 400 is shown. The computer system 400 can include a set of instructions that can be executed to cause the computer system 400 to perform any one or more of the methods or computer-based functions disclosed herein. The computer system 400 may operate as a standalone device or may be connected, e.g., using a network, to other computer systems or peripheral devices. Any of the components discussed above, such as the processor 150, may be a computer system 400 or a component in the computer system 400. In an embodiment, the computer system 400 involves a custom combination of discrete circuit components. The computer system 400 may implement embodiments for annotating a dataset of an authorized computer system 100.

For example, the instructions 412 may be operable when executed by the processor 402 to cause the computer 400 to access a database, such as database 102 of the authorized computer system 100, the database having a plurality of data items, each data item having one or more properties, each property of the one or more properties having an associated value, the database being structured with a pre-defined data model or format. The instructions 412 may also be operable to cause the processor 402 to access a dataset, such as dataset 104 of the authorized computer system 100, where a portion of the text contains data derived from the database. The instructions 412 may also be operable when executed by the processor 402 to cause the computer 400 to segment the text of the first dataset into tokens, the tokens having one or more characters, identify tokens in the first dataset that match property values in the database for predetermined database properties, determine whether the identified tokens in the first dataset represent values associated with a property in the database, annotate the identified tokens of the first dataset when the identified tokens are determined to represent values associated with a property in the database, and store the annotations and associated database properties and database values in a memory as an annotated dataset.

In a networked deployment, the computer system 400 may operate in the capacity of a server or as a client user computer in a client-server user network environment, or as a peer computer system in a peer-to-peer (or distributed) network environment. The computer system 400 can also be implemented as or incorporated into various devices, such as a personal computer (PC), a tablet PC, a set-top box (STB), a personal digital assistant (PDA), a mobile device, a palmtop computer, a laptop computer, a desktop computer, a communications device, a wireless telephone, a land-line telephone, a control system, a camera, a scanner, a facsimile machine, a printer, a pager, a personal trusted device, a web appliance, a network router, switch or bridge, or any other machine capable of executing a set of instructions (sequential or otherwise) that specify actions to be taken by that machine. In a particular embodiment, the computer system 400 can be implemented using electronic devices that provide voice, video or data communication. Further, while a single computer system 400 is illustrated, the term “system” shall also be taken to include any collection of systems or sub-systems that individually or jointly execute a set, or multiple sets, of instructions to perform one or more computer functions.

As illustrated in FIG. 4, the computer system 400 may include a processor 402, e.g., a central processing unit (CPU), a graphics processing unit (GPU), or both. The processor 402 may be a component in a variety of systems. For example, the processor 402 may be part of a personal computer or a workstation. The processor 402 may be one or more general processors, digital signal processors, application specific integrated circuits, field programmable gate arrays, servers, networks, digital circuits, analog circuits, combinations thereof, or other now known or later developed devices for analyzing and processing data. The processor 402 may implement a software program, such as code generated manually (i.e., programmed).

In an embodiment, single or multiple processors may be provided. Documents of the dataset 104 may be sent or received from different client computers over a data communication network. The computer system 400 may include a memory 404 that can communicate via a bus 408. The memory 404 may be a main memory, a static memory, or a dynamic memory. The memory 404 may include, but is not limited to computer readable storage media such as various types of volatile and non-volatile storage media, including but not limited to random access memory, read-only memory, programmable read-only memory, electrically programmable read-only memory, electrically erasable read-only memory, flash memory, magnetic tape or disk, optical media and the like. In one embodiment, the memory 404 includes a cache or random-access memory for the processor 402. In alternative embodiments, the memory 404 is separate from the processor 402, such as a cache memory of a processor, the system memory, or other memory. The memory 404 may be an external storage device or database for storing data. Examples include a hard drive, compact disc (“CD”), digital video disc (“DVD”), memory card, memory stick, floppy disc, universal serial bus (“USB”) memory device, or any other device operative to store data. The memory 404 is operable to store instructions executable by the processor 402. The functions, acts or tasks illustrated in the figures or described herein may be performed by the programmed processor 402 executing the instructions 412 stored in the memory 404. The functions, acts or tasks are independent of the particular type of instructions set, storage media, processor or processing strategy and may be performed by software, hardware, integrated circuits, firmware, micro-code and the like, operating alone or in combination. Likewise, processing strategies may include multiprocessing, multitasking, parallel processing and the like.

As shown, the computer system 400 may further include a display unit 414, such as a liquid crystal display (LCD), an organic light emitting diode (OLED), a flat panel display, a solid state display, a cathode ray tube (CRT), a projector, a printer or other now known or later developed display device for outputting determined information. The display 414 may act as an interface for the user to see the functioning of the processor 402, or specifically as an interface with the software stored in the memory 404 or in the drive unit 406.

Additionally, the computer system 400 may include an input device 416 configured to allow a user to interact with any of the components of system 400. The input device 416 may be a number pad, a keyboard, or a cursor control device, such as a mouse, or a joystick, touch screen display, remote control or any other device operative to interact with the system 400. In an embodiment, the input device 416 may facilitate a user in specifying a dataset 104 of the authorized computer system 100. For example, the display 414 may provide a listing of data in either of the database 102 or the dataset 104 of the authorized computer system 100. Further the input device 416 may allow for the selection of one or database property values to be annotated.

In a particular embodiment, as depicted in FIG. 4, the computer system 400 may also include a disk or optical drive unit 406. The disk drive unit 406 may include a computer-readable medium 410 in which one or more sets of instructions 412, e.g. software, can be embedded. Further, the instructions 412 may embody one or more of the methods or logic as described herein. In a particular embodiment, the instructions 412 may reside completely, or at least partially, within the memory 404 and/or within the processor 402 during execution by the computer system 400. The memory 404 and the processor 402 also may include computer-readable media as discussed above.

The present disclosure contemplates a computer-readable medium that includes instructions 412 or receives and executes instructions 412 responsive to a propagated signal, so that a device connected to a network 420 can communicate voice, video, audio, images or any other data over the network 420. Further, the instructions 412 may be transmitted or received over the network 420 via a communication interface 418. The communication interface 418 may be a part of the processor 402 or may be a separate component. The communication interface 418 may be created in software or may be a physical connection in hardware. The communication interface 418 is configured to connect with a network 420, external media, the display 414, or any other components in system 400, or combinations thereof. The connection with the network 420 may be a physical connection, such as a wired Ethernet connection or may be established wirelessly as discussed below. Likewise, the additional connections with other components of the system 400 may be physical connections or may be established wirelessly. In an embodiment, the communication interface 418 may be configured to communicate datasets with user devices.

The network 420 may include wired networks, wireless networks, or combinations thereof. The wireless network may be a cellular telephone network, an 802.11, 802.16, 802.20, or WiMAX network. Further, the network 420 may be a public network, such as the Internet, a private network, such as an intranet, or combinations thereof, and may utilize a variety of networking protocols now available or later developed including, but not limited to TCP/IP based networking protocols.

Embodiments of the subject matter and the functional operations described in this specification can be implemented in digital electronic circuitry, or in computer software, firmware, or hardware, including the structures disclosed in this specification and their structural equivalents, or in combinations of one or more of them. Embodiments of the subject matter described in this specification can be implemented as one or more computer program products, i.e., one or more modules of computer program instructions encoded on a computer readable medium for execution by, or to control the operation of, data processing apparatus. While the computer-readable medium is shown to be a single medium, the term “computer-readable medium” includes a single medium or multiple media, such as a centralized or distributed database, and/or associated caches and servers that store one or more sets of instructions. The term “computer-readable medium” shall also include any medium that is capable of storing, encoding or carrying a set of instructions for execution by a processor or that cause a computer system to perform any one or more of the methods or operations disclosed herein. The computer readable medium can be a machine-readable storage device, a machine-readable storage substrate, a memory device, or a combination of one or more of them. The term “data processing apparatus” or “data processing system” encompasses all apparatus, devices, and machines for processing data, including by way of example a programmable processor, a computer, or multiple processors or computers. The apparatus can include, in addition to hardware, code that creates an execution environment for the computer program in question, e.g., code that constitutes processor firmware, a protocol stack, a database management system, an operating system, or a combination of one or more of them.

In a particular non-limiting, exemplary embodiment, the computer-readable medium can include a solid-state memory such as a memory card or other package that houses one or more non-volatile read-only memories. Further, the computer-readable medium can be a random-access memory or other volatile re-writable memory. Additionally, the computer-readable medium can include a magneto-optical or optical medium, such as a disk or tapes or other storage device to capture carrier wave signals such as a signal communicated over a transmission medium. A digital file attachment to an e-mail or other self-contained information archive or set of archives may be considered a distribution medium that is a tangible storage medium. Accordingly, the disclosure is considered to include any one or more of a computer-readable medium or a distribution medium and other equivalents and successor media, in which data or instructions may be stored.

In an alternative embodiment, dedicated hardware implementations, such as application specific integrated circuits, programmable logic arrays and other hardware devices, can be constructed to implement one or more of the methods described herein. Applications that may include the apparatus and systems of various embodiments can broadly include a variety of electronic and computer systems. One or more embodiments described herein may implement functions using two or more specific interconnected hardware modules or devices with related control and data signals that can be communicated between and through the modules, or as portions of an application-specific integrated circuit. Accordingly, the present system encompasses software, firmware, and hardware implementations.

In accordance with various embodiments of the present disclosure, the methods described herein may be implemented by software programs executable by a computer system. Further, in an exemplary, non-limited embodiment, implementations can include distributed processing, component/object distributed processing, and parallel processing. Alternatively, virtual computer system processing can be constructed to implement one or more of the methods or functionality as described herein.

Although the present specification describes components and functions that may be implemented in particular embodiments with reference to particular standards and protocols, the invention is not limited to such standards and protocols. For example, standards for Internet and other packet switched network transmission (e.g., TCP/IP, UDP/IP, HTML, HTTP, HTTPS) represent examples of the state of the art. Such standards are periodically superseded by faster or more efficient equivalents having essentially the same functions. Accordingly, replacement standards and protocols having the same or similar functions as those disclosed herein are considered equivalents thereof.

A computer program (also known as a program, software, software application, script, or code) can be written in any form of programming language, including compiled or interpreted languages, and it can be deployed in any form, including as a standalone program or as a module, component, subroutine, or other unit suitable for use in a computing environment. A computer program does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

The processes and logic flows described in this specification can be performed by one or more programmable processors executing one or more computer programs to perform functions by operating on input data and generating output. The processes and logic flows can also be performed by, and apparatus can also be implemented as, special purpose logic circuitry, e.g., a reconfigurable logic device or an ASIC (application specific integrated circuit). As used herein, the terms “microprocessor” may refer to a hardware device that fetches instructions and data from a memory or storage device and executes those instructions (for example, an Intel Xeon processor or an AMD Opteron processor) to then, for example, process the data in accordance therewith. The term “reconfigurable logic” may refer to any logic technology whose form and function can be significantly altered (i.e., reconfigured) in the field post-manufacture as opposed to a microprocessor, whose function can change post-manufacture, e.g. via computer executable software code, but whose form, e.g. the arrangement/layout and interconnection of logical structures, is fixed at manufacture. The term “software” will refer to data processing functionality that is deployed on a computer. The term “firmware” will refer to data processing functionality that is deployed on reconfigurable logic. One example of a reconfigurable logic is a field programmable gate array (“FPGA”) which is a reconfigurable integrated circuit. An FPGA may contain programmable logic components called “logic blocks”, and a hierarchy of reconfigurable interconnects that allow the blocks to be “wired together”—somewhat like many (changeable) logic gates that can be inter-wired in (many) different configurations. Logic blocks may be configured to perform complex combinatorial functions, or merely simple logic gates like AND, OR, NOT and XOR. An FPGA may further include memory elements, which may be simple flip-flops or more complete blocks of memory. In an embodiment, processor 150 shown in FIG. 2 may be implemented using an FPGA or an ASIC. For example, the receiving, augmenting, communicating, and/or presenting may be implemented using the same FPGA.

Processors suitable for the execution of a computer program include, by way of example, both general and special purpose microprocessors, and anyone or more processors of any kind of digital computer. Generally, a processor will receive instructions and data from a read only memory or a random access memory or both. The essential elements of a computer are a processor for performing instructions and one or more memory devices for storing instructions and data. Generally, a computer will also include, or be operatively coupled to receive data from or transfer data to, or both, one or more mass storage devices for storing data, e.g., magnetic, magneto optical disks, or optical disks. However, a computer need not have such devices. Moreover, a computer can be embedded in another device, e.g., a mobile telephone, a personal digital assistant (PDA), a mobile audio player, a Global Positioning System (GPS) receiver, to name just a few. Computer readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto optical disks; and CD ROM and DVD-ROM disks. The processor and the memory can be supplemented by, or incorporated in, special purpose logic circuitry.

To provide for interaction with a user, embodiments of the subject matter described in this specification can be implemented on a device having a display, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input.

Embodiments of the subject matter described in this specification can be implemented in a computing system that includes a back end component, e.g., as a data server, or that includes a middleware component, e.g., an application server, or that includes a front end component, e.g., a client computer having a graphical user interface or a Web browser through which a user can interact with an implementation of the subject matter described in this specification, or any combination of one or more such back end, middleware, or front end components. The components of the system can be interconnected by any form or medium of digital data communication, e.g., a communication network. Examples of communication networks include a local area network (“LAN”) and a wide area network (“WAN”), e.g., the Internet.

The computing system can include clients and servers. A client and server are generally remote from each other and typically interact through a communication network. The relationship of client and server arises by virtue of computer programs running on the respective computers and having a client-server relationship to each other.

In an embodiment, the exemplary framework disclosed herein may apply to automatically creating a large annotated corpus, or text-based data set, which may then be used to train automated, machine learning, or artificial intelligence-based systems/models for identifying PHI in other data sets. To accomplish this, the exemplary framework needs access to both a database known to contain PHI and a corpus of data (i.e., an unannotated dataset). It is assumed that the data in the corpus is, at least in part, derived from the data in the database. For example, the data may have first existed in the database and then was copied over to a document in the corpus, such as an email, note, or file. An exemplary database known to contain PHI is shown below in Table 1.

TABLE 1
Example database

| gender | first | last | age | pulse | state | ssn |
|---|---|---|---|---|---|---|
| M | Jack | Green | 32 | 63 | CA | 905442410 |
| F | Sue | Cook | 63 | 80 | CA | 394502477 |
| M | Han | Morgan | 71 | 105.2 | CA | 832550554 |
| F | Noel | Wood | 72 | 86 | IL | 739946539 |
| F | Brittney | Smith | 36 | 82 | MN | 378347392 |
| M | Bob | Dole | 99 | 60 | CA | 947218403 |
| F | Jennifer | Anderson | 6 | 65 | CA | 104757333 |

As shown above, Table 1 contains medical information about patients. Since the disclosed framework in this example operates on a document, such as JSON and XML formatted files, the term item, which is an entity in the database described by the document, will be used in place of a row in a database since the term item could also be a triple in a triple store. In the case of a semantic web, as will be discussed below with respect to FIG. 5, an item or entity is the central item in a triple store. In this example, Table 1 may be a relational database, where each row of the database is an item, and a property of the item is a related value that appears in the document as a parsed token from the text or structured data file. As discussed above, a name space may be incorporated into the name of a property since there is no hierarchy to them. For example, the property “patient.age” could represent a column “age” in table “patient” found in an RDBMS. In this example, the fourth row of Table 1 represents a patient item with first name “Noel” as a property of this item.

FIG. 5 illustrates an exemplary relationship between database items, properties, and values. In FIG. 5, the left side represents a RDBMS 500 and the right side represents a semantic web 502. For the RDBMS 500, an item 504 may be the circled row containing properties 506 “gender,” “first,” “last,” and “age.” In this example, the associated values 508 of the properties 506 are “M,” “Jack,” “Green,” and “32,” respectively. For the semantic web 502, an item 504 is the central data item “<id: 904>” with the properties 506 “<age>,” “<first>,” “<last>,” and “<gender>” surrounding the central item 504. The values 508 for the semantic web 502 are the same as those mentioned above for the RDBMS 500.

FIG. 6 illustrates an exemplary annotated document segment from a corpus. In this example, it is assumed that a doctor created the document segment of FIG. 6 from the example database in Table 1. FIG. 6 is an example of what annotations 600 the tagger (the disclosed framework, or software, that annotates the un-annotated corpus) would assign. Each annotation is assigned at least the probability that the token originates from the database along with text span offsets in the document, along with associated properties and values, as shown in Table 2 below. The annotations may contain other information as well.

TABLE 2
Example annotation results

| value | property | start | end | probability |
|---|---|---|---|---|
| Noel | first | 50 | 53 | 0.956 |
| Wood | last | 55 | 58 | 0.895 |
| 72 | age | 77 | 78 | 0.631 |

While annotating natural language documents or structured data, the disclosed framework, or algorithm, assigns a probability of the token (word or symbol, usually delimited by white space) belonging to the database. The algorithm assigns information to answer the following for each parsed token: a) is the token in the database, b) if the token is in the database does this mention of it come from the database, and c) if the token comes from the database, what item and property does it belong to.

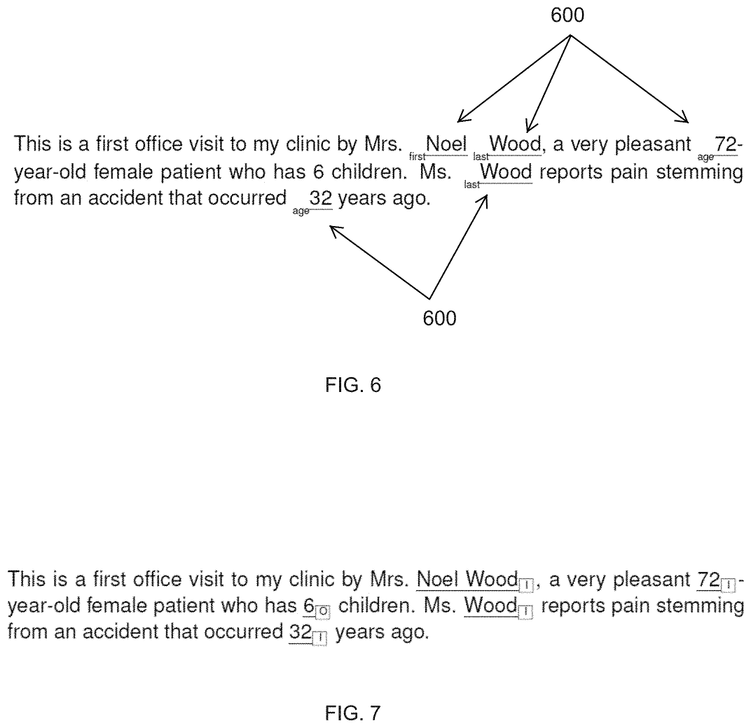

FIG. 7 illustrates other annotations to the exemplary document segment of FIG. 6. In this example, the annotations “I” indicate that a token is in the database and “O” indicates the token is out of the database. In this case, the name and age tokens (such as “Noel Wood” and “72”) are annotated as being in the database (i.e., originate from the database) whereas the token “6” referring to a number of children is annotated as being out of the database (i.e., not originating from the database). Even though the number “6” may be found in the database, it should not be annotated since the token describes a number of children rather than an age, which is what the number “6” represents in the exemplary database of Table 1 (as the age value for the item relating to patient Jennifer Anderson).

The process of the exemplary framework will now be discussed with reference to the example database shown in Table 1.

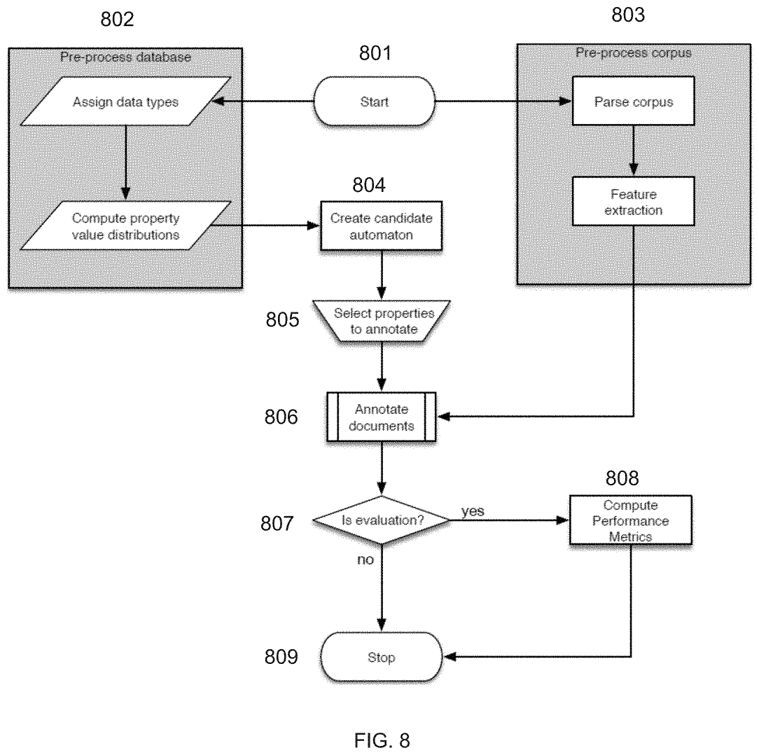

FIG. 8 depicts a flow chart illustrating an overview of an exemplary process. At the start 801 of the process, the exemplary framework pre-processes the database 802 and pre-processes the corpus 803. In one example, the exemplary framework may pre-process the database 802 and corpus 803 at the same time. In another example, the database may be pre-processed 802 first. In yet another example, pre-processing the database 802 may not occur. To pre-process the database 802, the exemplary framework may assign data types and compute property value distributions, as discussed above with respect to FIGS. 1B and 3. Referring to the exemplary database of Table 1 for example, the chance the string “IL” comes from the “State” property (without prior knowledge) is P (d ∈ ψ={CA, IL, NM})={2/7, 6/7, 6/7}. To pre-process the corpus 803, the exemplary framework may parse the corpus and extract features, as discussed above with respect to FIG. 1B and 3.

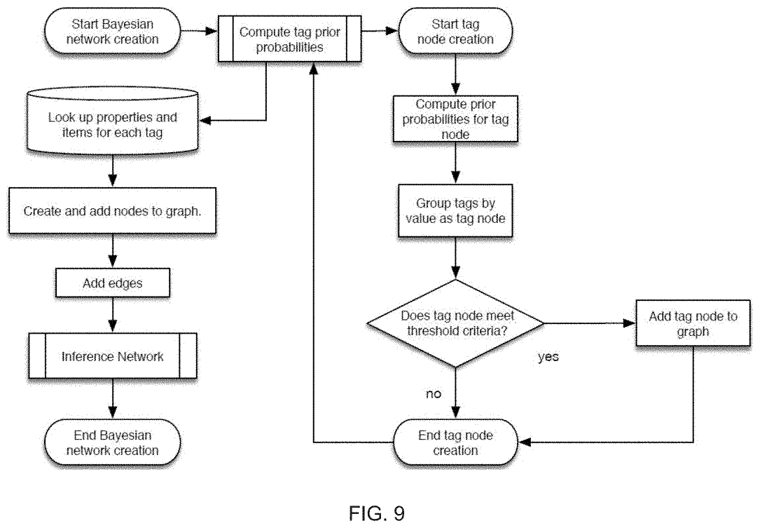

Once the database and corpus are prepared, the exemplary framework creates candidate automation 804 (i.e., identifying tokens that match property values in the database) and selects properties to annotate 805, as discussed above with respect to FIG. 1B and 3. To select properties to annotate 805, the exemplary framework creates the Bayesian network and dependencies in order to determine whether identified tokens are derived from the database. FIG. 9 depicts a flow chart illustrating an overview of creating an exemplary Bayesian network. The Bayesian network is created using an inferencing process, as discussed above with reference to FIG. 2.

FIG. 10 illustrates an exemplary Bayesian network constructed using the data of the exemplary database of Table 1 according to the process shown in FIG. 9. As shown in FIG. 10, each token, property, and item are represented as nodes in the graph. For example, the token nodes 1010 are the uppermost nodes in the Bayesian network and include values of “63,” “72,” “86,” “Noel,” “F,” “Wood,” and “739946539.” Property nodes 1020 are the children nodes of the parent token nodes 1010 and include values of “age,” “pulse,” “first,” “gender,” “last,” and “ssn.” Item nodes 1030 are the children nodes of the parent property nodes 1020 and include values of “Britney Smith,” “Jack Green,” “Jennifer Anderson,” “Noel Wood,” and “Sue Cook.” The bottommost child node of the Bayesian network is the match node 1040. The match node 1040 ties all item nodes 1030 together. As discussed above, a CPT for each property is based on the parent binary token node 1010 variables with probabilities over those parent tokens and the null hypothesis state. For example, the “age” property node 1020 has two parents (the “63” and “72” token nodes 1010) with three posterior states (63, 72, and the null hypothesis). An exemplary CPT for the “pulse” property node 1020 of FIG. 10 is shown below in Table 3.

TABLE 3
Example Property Node Conditional Probability Table

| Parents | 86 | 63 | Null hypothesis |
|---|---|---|---|
| 86 = out, 63 = out | 0 | 0 | 1 |
| 86 = out, 63 = in | 0 | 0.857143 | 0.142857 |
| 86 = in, 63 = out | 0.857143 | 0 | 0.142857 |
| 86 = in, 63 = in | 0.5 | 0.5 | 0 |

As discussed above, each item node represents a binary random variable, each binary random variable having a CPT parameterized by states of the parent property nodes 1020. Thus, the item nodes 1020 are the Cartesian products of those properties. Two rows of an exemplary CPT for an item node 1030 of FIG. 10 is shown below in Table 4.

TABLE 4
Example Two Rows of Item Node Conditional Probability Table

| Parents | Present | Absent |
|---|---|---|
| ssn = < . . . >, pulse, = 86, last = Wood, | 0 | 1 |
| gender = F, first = Noel, age = 63 | | |
| ssn = < . . . >, pulse = 86, last = Wood, | 1 | 0 |
| gender = F, first = Noel, age = 72 | | |

After the Bayesian network is created it is ready to be used for inferencing. The fully constructed graph structure, with no nodes observed, is provided in FIG. 10. At this point, known algorithms may be used to identify to which the most likely item the data belongs, identify to which properties related to one or more items the data belongs, assign posterior probabilities, and filter tokens, as discussed above. In one example, a high-performance loopy belief propagation algorithm, such as, for example, Pomegranate, may be used. The posterior probabilities are then updated in an iterative process as various nodes are observed (i.e., maximize the probability of a state on a Bayesian network node). Each step may give a more constrained view of what originated from the database by filtering based on probability thresholds, or hyperparameters, as discussed above.

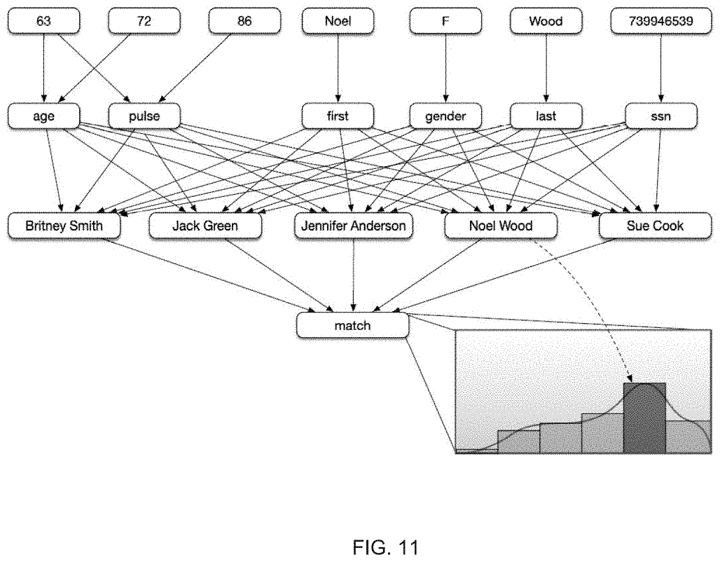

To begin the iterative Bayesian inferencing process, an item node 1030 needs to be selected in the Bayesian network graph. To do so, the exemplary process starts with the match node 1040 after the belief propagation algorithm finishes. In this example, the match node 1040 has a probability distribution of the items and the null hypothesis as seen in FIG. 11. The graph of the probability distribution in the bottom right of FIG. 11 represents how item node 1030 “Noel Wood” has a noticeable higher posterior probability than the other items in this example. The dotted arrow to the distribution graph represents which item defines the maximum probability of all items.

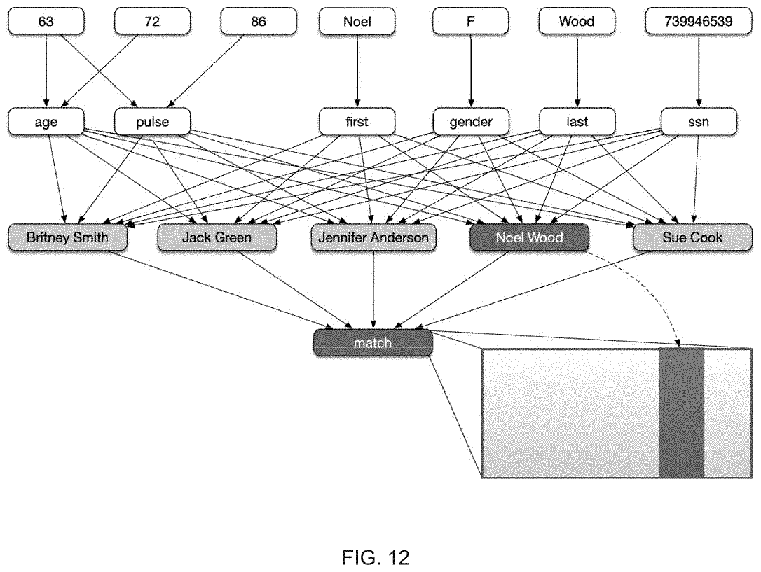

Using the MAP (maximum a posterior probability estimate) of the distribution over the observations in the match node, excluding the null hypothesis, the exemplary framework observes the highest probability item as being in the database, and all other items as not being in the database. In this example, that means observing the “Noel Wood” item node 1030 with the state of belonging to the database and observing the other items as not in the database. As shown in FIG. 12, the lighter nodes are observed to be the out of database state and the dark nodes in.

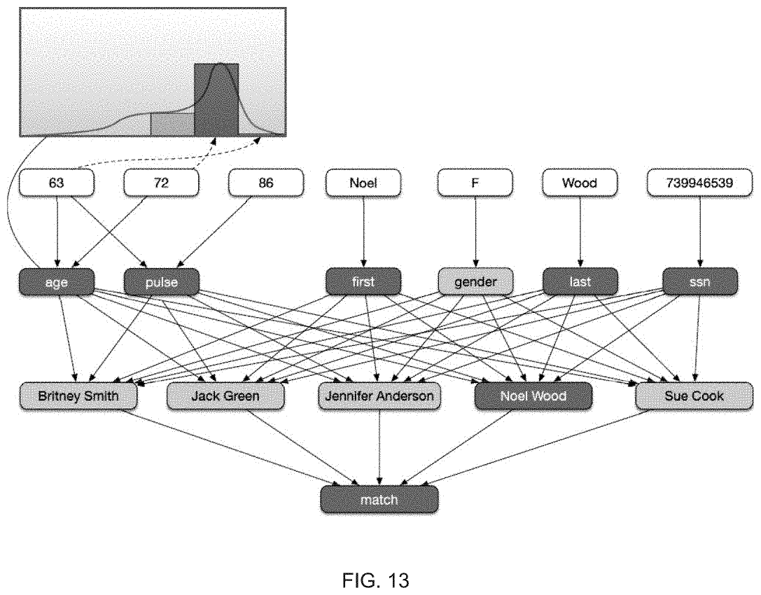

Next, the exemplary framework selects a property node 1020 in the Bayesian network graph. Once the match node 1040 and item nodes 1030 are observed, the Bayesian network loopy belief algorithm is rerun and the posterior probabilities recalculated. Each property node 1020 gets a new posterior probability, which represents how likely those properties are to be found in the database. Any properties that have a posterior probability higher than the property membership threshold (K_{ψ}) hyperparameter are modeled as those that belong to the database and are observed belonging to the database state, while the others are observed as out of the database using the null hypothesis state. In this example, the property “age” has a probability distribution of the null hypothesis=0:19, value “72”=0:81, and value “63”=0. The value “63” gets a zero probability because only the “Noel Wood” node has been observed. Another way of looking at it is that a non-zero probability is an inconsistent state of the graph. Because value “72” has a probability posterior estimate higher than K_{ψ}=0:4 its state is observed and the property considered as originating from the database. The same is done for the remaining properties as seen in FIG. 13, where the dark gray represents those properties with some parent state observed and light gray represents the null hypothesis state observed.

Next, the exemplary framework selects a token node 1010 in the Bayesian network graph. Once again, the Bayesian network loopy belief algorithm is rerun and the posterior probabilities recalculated with the token node 1010 posterior probabilities changing again. The token node 1010 posterior probabilities are used as the estimates for each token they represent. Similar to the process for the property nodes 1020, the posterior estimates of the token nodes 1010 are thresholded with hyperparameter tag membership threshold (Kₜ). Those token nodes 1010 that meet this criterion are considered as token nodes 1010 belonging to the database.

The example given above was for the “Noel Wood” item node 1030. However, the exemplary framework may repeat the process above for each item node 1030 in the Bayesian network graph. This is known as computing the full joint, which is the probability estimate of a graph for which every node has some observed state (i.e., a fully observed graph). The number of full joint estimates is a function of the nodes and the cardinality of the number of states for each node. Computing the full joint may be useful for considering multiple combinations of items. For example, in the exemplary process described above, the item node 1030 “Noel Wood” was selected because it was statistically significantly higher than the other item nodes 1030. However, if there was no statistical significance between item node 1030 “Noel Wood” and item node 1030 “Sue Cook,” the exemplary framework could iterate over the Bayesian network graph for each item nodes 1030 “Noel Wood” and “Sue Cook” and compute the full joint for both, and use the higher MAP. In order to compute the full joint, only one additional step to the exemplary process discussed above, which is to observe the token nodes 1010 by assigning them as being in the database state for those token nodes 1010 that meet the criteria for Kₜ.

Referring back to FIG. 8, once the exemplary framework selects properties to annotate 805 based on the determination of whether identified tokens are derived from the database using the Bayesian network, the documents are annotated 806. That is, after all token node 1010 posterior probabilities have been calculated as described above, the documents in the dataset are processed. All token nodes 1010 are then filtered based on Kₜ as detailed above. Tokens represented by the remaining token nodes 1010 are used to reference or identify all tokens in the documents. Each token has a character offset recorded when it was parsed, or segmented, from the document and that offset is now used to record the annotation in association with at least the token posterior probability estimate, the associated property value, and the associated item value, for which multiple are possible in case of calculating the full joint as described above. An annotation token level joining of text spans is done after all annotations are created. Specifically, if a token has another token with the same annotation information, one of the annotations is removed and the other annotation's text span is expanded to the span of the removed annotation.

The illustrations of the embodiments described herein are intended to provide a general understanding of the structure of the various embodiments. The illustrations are not intended to serve as a complete description of all of the elements and features of apparatus and systems that utilize the structures or methods described herein. Many other embodiments may be apparent to those of skill in the art upon reviewing the disclosure. Other embodiments may be utilized and derived from the disclosure, such that structural and logical substitutions and changes may be made without departing from the scope of the disclosure. Additionally, the illustrations are merely representational and may not be drawn to scale. Certain proportions within the illustrations may be exaggerated, while other proportions may be minimized. Accordingly, the disclosure and the figures are to be regarded as illustrative rather than restrictive.

While this specification contains many specifics, these should not be construed as limitations on the scope of the invention or of what may be claimed, but rather as descriptions of features specific to particular embodiments of the invention. Certain features that are described in this specification in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or variation of a sub-combination.

Similarly, while operations are depicted in the drawings and described herein in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the embodiments described above should not be understood as requiring such separation in all embodiments, and it should be understood that the described program components and systems can generally be integrated together in a single software product or packaged into multiple software products.

Reference in this specification to “one embodiment” or “an embodiment” means that a particular feature, structure, or characteristic described in connection with the embodiments is included in at least one embodiment of the disclosure. The appearances of the phrase “in one embodiment” in various places in the specification are not necessarily all referring to the same embodiment, nor are separate or alternative embodiments mutually exclusive of other embodiments. Moreover, various features are described which may be exhibited by some embodiments and not by others. Similarly, various requirements are described which may be requirements for some embodiments but not other embodiments.

One or more embodiments of the disclosure may be referred to herein, individually and/or collectively, by the term “invention” merely for convenience and without intending to voluntarily limit the scope of this application to any particular invention or inventive concept. Moreover, although specific embodiments have been illustrated and described herein, it should be appreciated that any subsequent arrangement designed to achieve the same or similar purpose may be substituted for the specific embodiments shown. This disclosure is intended to cover any and all subsequent adaptations or variations of various embodiments. Combinations of the above embodiments, and other embodiments not specifically described herein, will be apparent to those of skill in the art upon reviewing the description.

The Abstract of the Disclosure is provided to comply with 37 C.F.R. § 1.72(b) and is submitted with the understanding that it will not be used to interpret or limit the scope or meaning of the claims. In addition, in the foregoing Detailed Description, various features may be grouped together or described in a single embodiment for the purpose of streamlining the disclosure. This disclosure is not to be interpreted as reflecting an intention that the claimed embodiments require more features than are expressly recited in each claim. Rather, as the following claims reflect, inventive subject matter may be directed to less than all of the features of any of the disclosed embodiments. Thus, the following claims are incorporated into the Detailed Description, with each claim standing on its own as defining separately claimed subject matter.

It is therefore intended that the foregoing detailed description be regarded as illustrative rather than limiting, and that it be understood that it is the following claims, including all equivalents, that are intended to define the spirit and scope of this invention.

What is claimed is:

1. A computer implemented method comprising:
accessing, by a processor, a database stored in a memory, the database comprising a plurality of data items, each data item comprising one or more properties, each property of the one or more properties having an associated value, the database being structured with a pre-defined data model or format;
accessing, by the processor, a first dataset stored in the memory and comprising text, wherein a portion of the text contains data derived from the database;
segmenting, by the processor, the text of the first dataset into tokens, the tokens comprising one or more characters;
identifying, by the processor, tokens in the first dataset that match property values in the database for predetermined database properties;
determining, by the processor, whether the identified tokens in the first dataset represent values associated with a property in the database;
annotating, by the processor, the identified tokens of the first dataset when the identified tokens are determined to represent values associated with a property in the database, wherein annotating comprises associating a tag with each identified token and assigning annotation attributes for each tag; and
storing, by the processor, the annotations and associated database properties and database values in the memory as an annotated dataset.

2. The computer implemented method of claim 1, wherein the first dataset comprises a plurality of electronic documents relating to a plurality of patients.

3. The computer implemented method of claim 1, wherein the database and the first dataset are proprietary to an entity authorized under regulatory guidelines to possess the data in the database and the first dataset.

4. The computer implemented method of claim 1, wherein the text of the first dataset is unstructured without a pre-defined data model or format.

5. The computer implemented method of claim 1, wherein the data derived from the database contains protected health information.

6. The computer implemented method of claim 1, wherein the identifying of tokens in the first dataset comprises detecting tokens using a string searching algorithm.

7. The computer implemented method of claim 1, wherein the determining comprises:
calculating, by the processor, a prior probability, for each identified token, of whether the identified token represents a value associated with a property in the database based on a prevalence of the identified token in a second dataset;
iteratively calculating, by the processor, a posterior probability, for each identified token, of whether the identified token represents a value associated with a property in the database based on a Bayesian network, wherein the iterative calculating starts with observing a bottommost child node of the Bayesian network having the highest calculated prior probability and repeats for each layer of parent nodes of the child node of the Bayesian network; and
determining, by the processor, whether a respective identified token represents a value associated with a property in the database based on the calculated posterior probability for an uppermost parent node representing the respective identified token.

8. The computer implemented method of claim 7, wherein iteratively calculating comprises refining the calculated prior probability based on observing nodes for each layer of parent nodes of the Bayesian network and filtering refined prior probabilities based on predetermined probability thresholds.

9. The computer implemented method of claim 8, wherein observing nodes of the Bayesian network comprises maximizing the probability of a state on a Bayesian network node.

10. The computer implemented method of claim 1, wherein the annotation attributes include identification of database data items, database properties, database property values, a probability that the identified tokens represent values associated with a property in the database, a determination of whether the identified tokens represent values associated with a property in the database, character span information for characters of the identified tokens, or combinations thereof.

11. The computer implemented method of claim 7, wherein the first dataset and the second dataset are mutually exclusive.

12. The computer implemented method of claim 1, further comprising training a machine learning model using the annotated dataset, wherein the result is a machine learned model.

13. The computer implemented method of claim 12, further comprising identifying text in another dataset using the machine learned model.

14. An automatic annotating system comprising:
a data preparer configured to access, from an authorized system, a database and a first dataset stored in a memory, the database comprising a plurality of data items, each data item comprising one or more properties, each property of the one or more properties having an associated value, the first dataset comprising text, wherein a portion of the text contains data derived from the database;
a tokenizer coupled with the data preparer and configured to segment the text of the first dataset into tokens, the tokens comprising one or more characters;
a data analyzer coupled with the tokenizer and configured to identify tokens in the first dataset that match property values in the database for predetermined database properties and determine whether the identified tokens in the first dataset represent values associated with a property in the database; and
an annotator coupled with the data analyzer and configured to annotate the identified tokens of the first dataset when the identified tokens are determined to represent values associated with a property in the database, wherein the annotator, to annotate the identified tokens, is further configured to associate a tag with each identified token and assign annotation attributes for each tag,
wherein the respective tags, the identified tokens associated with the respective tags, and the assigned annotation attributes for the respective tags are stored in the memory as an annotated dataset.

15. The automatic annotating system of claim 14, wherein the first dataset comprises a plurality of electronic documents relating to a plurality of patients and wherein the data derived from the database contains protected health information.

16. The automatic annotating system of claim 14, wherein the data analyzer is further configured to:
calculate a prior probability, for each identified token, of whether the identified token represents a value associated with a property in the database based on a prevalence of the identified token in a second dataset;
iteratively calculate a posterior probability, for each identified token, of whether the identified token represents a value associated with a property in the database based on a Bayesian network, wherein the iterative calculating starts with observing a child node of the Bayesian network having the highest calculated prior probability and repeats for each layer of parent nodes of the child node of the Bayesian network; and
determine whether a respective identified token represents a value associated with a property in the database based on the calculated posterior probability for the respective identified token.

17. The automatic annotating system of claim 16, wherein the data analyzer is further configured to adjust the calculated prior probability, wherein, to adjust the calculated prior probability, the data analyzer is configured to observe nodes for each layer of parent nodes of the Bayesian network and filter adjusted prior probabilities based on predetermined probability thresholds.

18. The automatic annotating system of claim 17, wherein, to observe nodes of the Bayesian network, the data analyzer is further configured to maximize the probability of a state on a Bayesian network node.

19. The automatic annotating system of claim 16, wherein the first dataset and the second dataset are mutually exclusive.

20. An automatic annotating system comprising:
a means for accessing a database, the database comprising a plurality of data items, each data item comprising one or more properties, each property of the one or more properties having an associated value;
a means for accessing a first dataset comprising text, wherein a portion of the text contains data derived from the database;
a means for segmenting the text of the first dataset into tokens, the tokens comprising one or more characters;
a means for identifying tokens in the first dataset that match property values in the database for predetermined database properties;
a means for determining whether the identified tokens in the first dataset represent values associated with a property in the database;
a means for annotating the identified tokens of the first dataset when the identified tokens are determined to represent values associated with a property in the database, wherein annotating comprises associating a tag with each identified token and assigning annotation attributes for each tag; and
a means for storing the annotations and associated database properties and database values in a memory as an annotated dataset.
